(19) United States
(12) Patent Application Publication
(10) Pub. No.: US 2021/0284758 A1
(43) Pub. Date: Sep. 16, 2021

(54) POLYMER-CYCLIC MOLECULE STRUCTURE AND METHOD FOR PRODUCING THE SAME

(71) Applicant: The University of Tokyo, Tokyo (JP)

(72) Inventors: Kohzo Ito, Tokyo (JP); Rina Maeda, Tokyo (JP); Shuntaro Uenuma, Tokyo (JP); Kimika Endo, Tokyo (JP)

(21) Appl. No.: 17/178,808

(22) Filed: Feb. 18, 2021

(30) Foreign Application Priority Data
Feb. 20, 2020 (JP) 2020-027578

(51) Int. Cl. C08B 37/16; A61K 47/61; A61K 9/50

(52) U.S. Cl. CPC C08B 37/0015; A61K 47/61; B82Y 5/00; A61K 9/5031; A61K 9/5036

(57) ABSTRACT

Provided is a structure including a plurality of pseudo-polyrotaxanes and/or polyrotaxanes each including a chain polymer included in a cavity or cavities of one or more cyclic molecules in a skewered manner, at least part of the plurality of pseudo-polyrotaxanes and/or polyrotaxanes being arranged in series with each other.

### CROSS-REFERENCE TO RELATED APPLICATION

The present application claims a benefit of priority from Japanese Patent Application No. 2020-027578 filed on Feb. 20, 2020, the entirety of which is incorporated herein by reference.

### BACKGROUND OF THE INVENTION

### 1. Field of the Invention

The present invention relates to a structure including a plurality of pseudo-polyrotaxanes and/or polyrotaxanes each including a chain polymer included in a cavity or cavities of one or more cyclic molecules in a skewered manner, and a method of producing the structure.

### 2. Description of the Related Art

In recent years, progress has been made in development of a nanosheet having a thickness of 100 nm or less into applications such as a drug, a catalyst, an optical material, an electrode, and a biomaterial. WO 2020/013215 A1 discloses an isolated nanosheet including a plurality of pseudo-polyrotaxanes and/or polyrotaxanes each including a linear molecule included in a cavity or cavities of one or more cyclic molecules, such as a cyclodextrin, in a skewered manner, in which the linear molecule includes, as part thereof, a first linear molecule having an ionizable group that ionizes in water or an aqueous solution.

### SUMMARY OF THE INVENTION

Such single-layer nanosheet as described in WO 2020/013215 A1 in which one pseudo-polyrotaxane and/or polyrotaxane molecule is arranged in its thickness direction is thin, and hence a substance such as a drug cannot be incorporated in a large amount into the sheet. Accordingly, it would be advantageous to design a structure in which the pseudo-polyrotaxanes and/or polyrotaxanes each including a chain polymer included in the cavity of the cyclic molecule in a skewered manner extend not only in parallel but also in series.

Although an increase in length of the chain polymer can increase the thickness of the sheet, one chain polymer needs to be included in many cyclic molecules, and the production of such pseudo-polyrotaxane and/or polyrotaxane requires large energy for a reaction, thereby leading to much cost and time.

A problem to be solved by the present invention is to provide a structure including a plurality of pseudo-polyrotaxanes and/or polyrotaxanes, the structure having a thickness larger than that of a related-art nanosheet, and a method of producing the structure.

The inventors of the present invention have made extensive investigations with a view to solving the problem, and as a result, have found that when the structure of a chain polymer for forming a pseudo-polyrotaxane and/or a polyrotaxane is designed so that both ends of the chain polymer may be housed in a columnar structure formed of a plurality of cyclic molecules, that is, a column without protruding therefrom, the plurality of pseudo-polyrotaxanes and/or polyrotaxanes are stacked in series by a noncovalent bond interaction between the adjacent cyclic molecules of the adjacent pseudo-polyrotaxanes and/or polyrotaxanes. Thus, the inventors have completed the present invention.

According to one aspect of the present invention, there is provided a structure including a plurality of pseudo-polyrotaxanes and/or polyrotaxanes each including a chain polymer included in a cavity or cavities of one or more cyclic molecules in a skewered manner, at least part of the plurality of pseudo-polyrotaxanes and/or polyrotaxanes being arranged in series with each other.

According to another aspect of the present invention, there is provided a method of producing a structure including a plurality of pseudo-polyrotaxanes and/or polyrotaxanes each including a chain polymer included in a cavity or cavities of one or more cyclic molecules in a skewered manner, the method including causing the plurality of pseudo-polyrotaxanes and/or polyrotaxanes each including the chain polymer having both ends housed in a column formed of the plurality of cyclic molecules to interact with each other to arrange at least part of the plurality of pseudo-polyrotaxanes and/or polyrotaxanes in series with each other.

### BRIEF DESCRIPTION OF THE DRAWINGS

FIG. 1A is a schematic view for illustrating a cyclic molecule and a chain polymer.

FIG. 1B is a schematic view for illustrating a pseudo-polyrotaxane and/or polyrotaxane unit.

FIG. 2 is a schematic perspective view of a structure according to at least one embodiment of the present invention formed by the aggregation of a plurality of pseudo-polyrotaxanes.

FIG. 3A, FIG. 3B, FIG. 3C, and FIG. 3D are each a schematic view for illustrating a relationship between the molecular weight of the chain polymer and the shape of the structure.

FIG. 3A is an illustration of the production example of a rod-shaped structure in which its PEO is a short chain.

FIG. 3B is an illustration of the production example of a cube-shaped structure in which its PEO is longer than that of FIG. 3A.

FIG. 3C is an illustration of the production example of a sheet-shaped structure in which its PEO is even longer than that of FIG. 3B.

FIG. 3D is an illustration of the production example of a single-layer nanosheet-shaped structure in which its PEO is even longer than that of FIG. 3C.

FIG. 4A, FIG. 4B, and FIG. 4C are illustrations of a branched chain polymer and the sheet of a structure using the polymer.

FIG. 4A is a schematic view for illustrating a branched PEO.

FIG. 4B is a schematic view of the sheet of the structure using the branched PEO of FIG. 4A.

FIG. 4C is a schematic view for illustrating the linked state of the sheet of FIG. 4B.

FIG. 5A, FIG. 5B, FIG. 5C, FIG. 5D, FIG. 5E, and FIG. 5F are views for illustrating a relationship between the order in which γ-CD is chemically bonded to portions (segments) in the chain polymer different from each other in composition and the shape of the structure.

FIG. 5A is an illustration of a tripolymer having three segments in which a central portion is a PPO and both ends are each a PEO having a molecular weight of 0.2k.

FIG. 5B is an illustration of a tripolymer having three segments in which a central portion is the PPO and both ends are each a PEO having a molecular weight of 1.1k.

FIG. 5C is an illustration of a tripolymer having three segments in which a central portion is the PPO and both ends are each a PEO having a molecular weight of 6.5k.

FIG. 5D is a schematic view (left) of a structure formed by using the chain polymer of FIG. 5A, and an enlarged view (right) of a portion surrounded with a circle.

FIG. 5E is a schematic view (left) of a structure formed by using the chain polymer of FIG. 5B, and an enlarged view (right) of a portion surrounded with a circle.

FIG. 5F is a schematic view (left) of a structure formed by using the chain polymer of FIG. 5C, and an enlarged view (right) of a portion surrounded with a circle.

FIG. 6A is a schematic front view of a pseudo-polyrotaxane whose chain polymer is a polypropylene oxide (PPO).

FIG. 6B is a schematic front view of a pseudo-polyrotaxane whose chain polymer is a polyethylene oxide (PEO).

FIG. 6C is a schematic front view of a pseudo-polyrotaxane whose chain polymer extends from one end of a column formed of a series of cyclic molecules to the other end thereof.

FIG. 6D is a schematic front view of a pseudo-polyrotaxane whose chain polymer extends beyond the lower end of a column formed of six cyclic molecules, but does not extend up to the upper end thereof.

FIG. 6E is a schematic front view of a pseudo-polyrotaxane whose chain polymer reaches none of the upper end and lower end of a column formed of six cyclic molecules.

FIG. 6F is a schematic front view for illustrating a series of cyclic molecules free of any chain polymer.

FIG. 7 is a schematic perspective view of a specific embodiment of a structure including a through-hole.

FIG. 8A is a photograph at the time of observation with naked eyes in the formation of PPR_EO 400.

FIG. 8B is a photograph obtained by observation with an optical microscope.

FIG. 8C is a photograph obtained by observation with a SEM.

FIG. 9A is a SEM image of the structure of the PPR_EO 400 (PEO having a weight-average molecular weight of 400).

FIG. 9B is a SEM image of the structure of PPR_EO 4k (PEO having a weight-average molecular weight of 4,000).

FIG. 9C is a SEM image of the structure of PPR_EO 6k (PEO having a weight-average molecular weight of 6,000).

FIG. 9D is a SEM image of the structure of PPR_EO 20k (PEO having a weight-average molecular weight of 20,000).

FIG. 10 is a WAXS profile of each of the PPR_EO 400, PPR_EO 2k, the PPR_EO 4k, the PPR_EO 6k, and the PPR_EO 20k.

FIG. 11A is a SEM image of the structure of PPR_PO 400 (PPO having a weight-average molecular weight of 400).

FIG. 11B is a SEM image of the structure of PPR_PO 2k (PPO having a weight-average molecular weight of 2,000).

FIG. 11C is a SEM image of the structure of PPR_PO 4k (PPO having a weight-average molecular weight of 4,000).

FIG. 12A is a SEM image of PPR_EO₄PO₅₆EO₄.

FIG. 12B is a SEM image of PPR_EO₂₅PO₅₆EO₂₅.

FIG. 12C is a SEM image of PPR_EO₁₄₇PO₅₆EO₁₄₇.

FIG. 12D is a WAXS profile in PPR_EO₁₄₇PO₅₆.

FIG. 12E is an AFM image and a height profile of the PPR_EO₁₄₇PO₅₆.

FIG. 13 is a microphotograph of a nanosheet including a through-hole.

FIG. 14 is a microphotograph of the microstructure of Example 6.

FIG. 15 is a microphotograph of the microstructure of Example 7.

FIG. 16A, FIG. 16B, FIG. 16C, and FIG. 16D are each a microphotograph of the microstructure of Example 8.

FIG. 17 is a microphotograph of the microstructure of Example 9.

FIG. 18 is a microphotograph of the microstructure of Example 10.

FIG. 19A and FIG. 19B are microphotographs of the microstructure of Example 11 at a low magnification and a high magnification, respectively. Scale bars each represent a length.

FIG. 20A, FIG. 20B, and FIG. 20C are microphotographs of the structure of Example 12 at a low magnification, a medium magnification (enlarging the portion of FIG. 20A surrounded with a dotted line), and a high magnification (enlarging the portion of FIG. 20B surrounded with a dotted line), respectively. Scale bars each represent a length.

FIG. 21 is a microphotograph of the structure of Example 13. An inserted figure on the upper right is an enlarged photograph.

FIG. 22A and FIG. 22B are each a microphotograph of the microstructure of Example 14. Scale bars each represent a length.

FIG. 22A is a phase-contrast microscope photograph.

FIG. 22B is a fluorescence microscope photograph.

FIG. 23A and FIG. 23B are microphotographs of the Skin_β-CD/EO₇₅PO₃₀EO₇₅ structure of Example 15 before its adhesion and after the adhesion, respectively.

FIG. 24A and FIG. 24B are microphotographs of the hair_β-CD/EO₇₅PO₃₀EO₇₅ structure of Example 15 before its adhesion and after the adhesion, respectively.

FIG. 25A and FIG. 25B are microphotographs of the lens_β-CD/EO₇₅PO₃₀EO₇₅ structure of Example 15 before its adhesion and after the adhesion, respectively.

FIG. 26 is a microphotograph of the eye_PPR_EO₁₄₇PO₅₆EO₁₄₇ structure of Example 15.

FIG. 27 is a graph of the absorbance of the β-CD/EO₇₅PO₃₀EO₇₅ structure of Example 16 with respect to the wavelength of incident light.

FIG. 28A is a SEM image of a porous membrane.

FIG. 28B is a SEM image of pore-β-CD/EO₇₅PO₃₀EO₇₅ (5 mL).

FIG. 28C is a SEM image of pore-β-CD/EO₇₅PO₃₀EO₇₅ (20 mL).

### DESCRIPTION OF THE EMBODIMENTS

Now, embodiments of the present invention are described with reference to the drawings.

The “polymer” as used herein refers to a compound formed of a molecule having a repeating structure derived from a monomer that is a monomeric substance. Although a chain polymer is preferably a single-chain polymer, when a cyclic molecule can move or rotate on the chain polymer, the chain polymer may be a polymer having a branch from its main chain.

The term “thickness direction of a structure” as used herein refers to a direction along the longitudinal direction of a pseudo-polyrotaxane and/or polyrotaxane unit, in other words, the longitudinal direction of a chain polymer for forming a pseudo-polyrotaxane and/or a polyrotaxane, and/or a direction along the axial direction of a cyclic molecule.

Herein, the formation of a structure can be recognized by small-angle X-ray scattering measurement, phase-contrast optical microscope observation, atomic force microscope observation, or scanning electron microscope observation.

The term “polyrotaxane” as used herein refers to a molecular assembly formed of one or more cyclic molecules and a chain polymer included in the cavity or cavities of the one or more cyclic molecules in a skewered manner, the molecular assembly having, at both terminals of the chain polymer, capping groups for preventing the one or more cyclic molecules from disengaging from the chain polymer. The term “pseudo-polyrotaxane” refers to a molecular assembly formed of one or more cyclic molecules and a chain polymer included in the cavity or cavities of the one ore more cyclic molecules in a skewered manner, the molecular assembly having the capping group at only one terminal of the chain polymer, or being free of the capping group at any one of the terminals of the chain polymer.

The term “cyclodextrin” as used herein refers to a cyclic oligosaccharide in which D-glucose undergoes α-1,4 glycosidic bonding to form a cyclic structure.

Herein, out of polyethylene oxides (PEOs), a PEO having a weight-average molecular weight of 20,000 or less is sometimes interchangeably referred to as “polyethylene glycol (PEG).”

Herein, out of polypropylene oxides (PPOs), a PPO having a weight-average molecular weight of 20,000 or less is sometimes interchangeably referred to as “polypropylene glycol (PPG).”

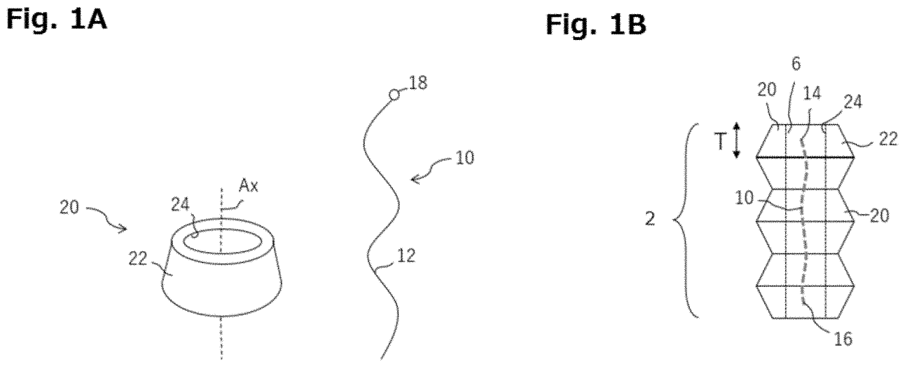

FIG. 1A is an illustration of a cyclic molecule 20 and a chain polymer 10. The cyclic molecule 20 includes: a main body 22 of a hollow and substantially truncated cone shape; and a cavity 24 defined and formed by the main body 22. The chain polymer 10 has a polymer main body 12, and in this example, has a modifying group 18 formed of an ionizable group or a non-ionizable group in one end portion of the polymer main body 12. FIG. 1B is an illustration of a pseudo-polyrotaxane and/or polyrotaxane 2 including the chain polymer 10 included in the cavity 24 of the cyclic molecule 20 in a skewered manner. In this figure, the chain polymer 10 extends to the inside of the cavity 24 of each of the six cyclic molecules 20, but both ends 14 and 16 of the chain polymer 10 do not reach both ends of a cavity 6, and a state in which the entirety of the chain polymer 10 is housed in the cavity 6 defined and formed by the six continuous cyclic molecules 20 is established.

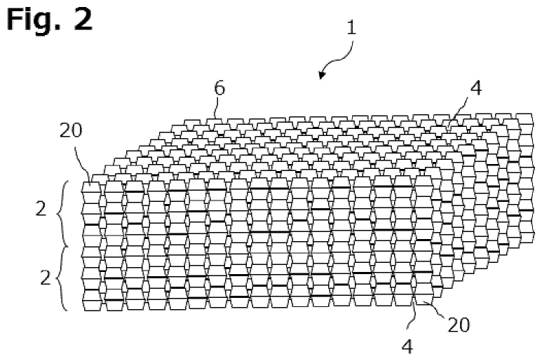

Without wishing to be bound by any theory, the cyclic molecules 20 present in the pseudo-polyrotaxane and/or polyrotaxane 2 illustrated in FIG. 1B aggregate in a columnar, that is, cylindrical manner to be columnized, and hence the outer surfaces thereof become hydrophobic. Accordingly, the plurality of pseudo-polyrotaxanes and/or polyrotaxanes 2 assemble with each other in a self-organized manner through the hydrophobic and columnized cyclic molecules 20 to form a structure 1 illustrated in at least one embodiment of FIG. 2. In this case, the plurality of cyclic molecules 20 aggregate for the one chain polymer 10, and hence the chain polymer 10 is stretched in a substantially linear manner, and steric hindrance between the chain polymers 10 is suppressed as compared to that in which the cyclic molecule 20 is not present on the chain polymer 10. Accordingly, the chain polymers 10 can be arranged at a higher density in the structure 1. The configuration of the structure 1 is described in detail later.

The inventors of the present invention have previously produced a separable nanosheet including a plurality of pseudo-polyrotaxanes and/or polyrotaxanes each including a chain polymer included in the cavity or cavities of one or more cyclic molecules in a skewered manner. However, the single-layer nanosheet in which one pseudo-polyrotaxane and/or polyrotaxane is arranged in its thickness direction is thin, and hence the nanosheet has involved, for example, a problem in that a substance such as a drug cannot be incorporated in a large amount into the sheet.

In view of the foregoing, this time, the inventors of the present invention have designed the structure of a chain polymer for forming a pseudo-polyrotaxane and/or a polyrotaxane so that both ends of the chain polymer may be housed in a columnar structure formed of a plurality of cyclic molecules, that is, a column. As a result, surprisingly, the inventors have found that the plurality of pseudo-polyrotaxanes and/or polyrotaxanes are stacked in series by a noncovalent bond interaction between the adjacent cyclic molecules of the adjacent pseudo-polyrotaxanes and/or polyrotaxanes. Examples of the noncovalent bond interaction include, but not limited to, an ionic bond, a hydrogen bond, and a van der Waals force.

Further, the inventors have attempted to control the structure of a structure including the plurality of pseudo-polyrotaxanes and/or polyrotaxanes by designing the structure of the chain polymer under various conditions. As a result, the inventors have found that the shape of the structure is controlled by changing the molecular weight, hydrophilicity and hydrophobicity, topology, and polymer blocks of the chain polymer.

Description is given by taking, as an example, a structure in which the cyclic molecule 20 is γ-cyclodextrin (hereinafter “γ-CD”) and the chain polymer 10 is a polyethylene oxide (PEO).

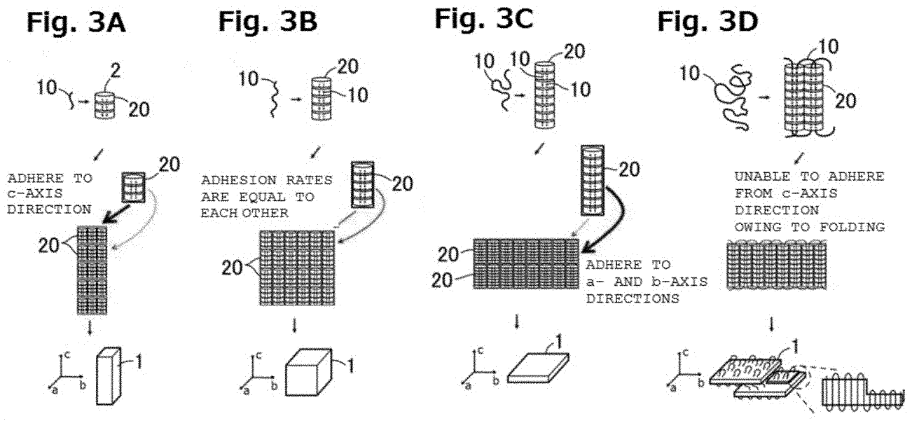

First, with regard to the effect of the molecular weight of the chain polymer, the dependence of the crystal growth of the structure on the length of a PEO axis is summarized in FIG. 3A to FIG. 3C. When the chain polymer 10 is the PEO, the cyclic molecule 20 forms a double-chain composite with the chain polymer 10.

As illustrated in FIG. 3A, when the chain polymer 10 has a small molecular weight and a short axis, a rod-shaped structure in which the pseudo-polyrotaxanes and/or polyrotaxanes 2 are stacked long in a c-axis direction, that is, a direction parallel to the main axis of the chain polymer 10 is formed. That is, the foregoing means that the crystal growth of the cyclic molecule 20 in the c-axis direction is faster than the crystal growth thereof in each of a-axis and b-axis directions perpendicular to the c-axis. In contrast, as the axis of the chain polymer 10 becomes longer, the side surface length of the structure in the c-axis direction becomes shorter. The foregoing means that in the longer chain polymer 10, the crystal growth of the cyclic molecule 20 along each of the a-axis and the b-axis is faster than that along the c-axis. It is assumed that when the length of the pseudo-polyrotaxane and/or polyrotaxane 2 is short, its interaction in a lateral direction is weaker than that of the longer pseudo-polyrotaxane and/or polyrotaxane 2, and hence the pseudo-polyrotaxane and/or polyrotaxane 2 extends in the c-axis direction.

In an example illustrated in FIG. 3B, when the length of the chain polymer 10 is made longer than that in FIG. 3A, the interaction in the lateral direction becomes larger than that in FIG. 3A, and hence a cube-shaped structure whose length in the c-axis direction, and lengths in the a-axis and b-axis directions are equal to each other is formed.

In an example illustrated in FIG. 3C, when the length of the chain polymer 10 is made even longer than that in FIG. 3B, and hence a sheet-shaped structure whose lengths in the a-axis and b-axis directions are longer than its length in the c-axis direction is formed.

When the length of the chain polymer 10 is made even longer than that in FIG. 3C, as illustrated in FIG. 3D, the chain polymer 10 starts to bend, and a bent site of the chain polymer 10 inhibits the crystal growth of the cyclic molecule 20 along the c-axis. Thus, a sheet-shaped structure is formed. A portion including the bent site of the chain polymer 10 protrudes from the cyclic molecule 20. When the length or molecular weight of the chain polymer 10 is changed as described above, the behavior of the crystal of the structure formed by the cyclic molecule 20 can be controlled.

Next, the effects of the hydrophilicity and hydrophobicity of the chain polymer 10 are described. When the cyclic molecule 20 is γ-CD and the chain polymer 10 is the PEO, as described above, structural bodies of various shapes are formed along with a change in molecular weight of the chain polymer. In contrast, when a polypropylene oxide (PPO) that is hydrophobic is used instead of the PEO that is hydrophilic as the chain polymer 10, as the chain polymer 10 becomes longer, the shape of the structure changes from a rod shape whose length in the c-axis direction is longer than its lengths in the a- and b-axis directions to a cube shape whose length in the c-axis direction is substantially equal to its lengths in the a- and b-axis directions, to a sheet shape whose length in the c-axis direction is shorter than its lengths in the a- and b-axis directions, and to a random (disordered) shape. As described above, the hydrophilicity and hydrophobicity of the chain polymer 10 may also affect the behavior of the crystal of the structure.

Without wishing to be bound by any theory, the above-mentioned phenomenon is considered as follows: when the chain polymer 10 is hydrophilic, hydration occurs on the surface of the structure to stabilize the structure thereof; and in contrast, when the chain polymer 10 is hydrophobic, its hydrophobic aggregation competes with the crystallization of γ-CD to make the structure disordered.

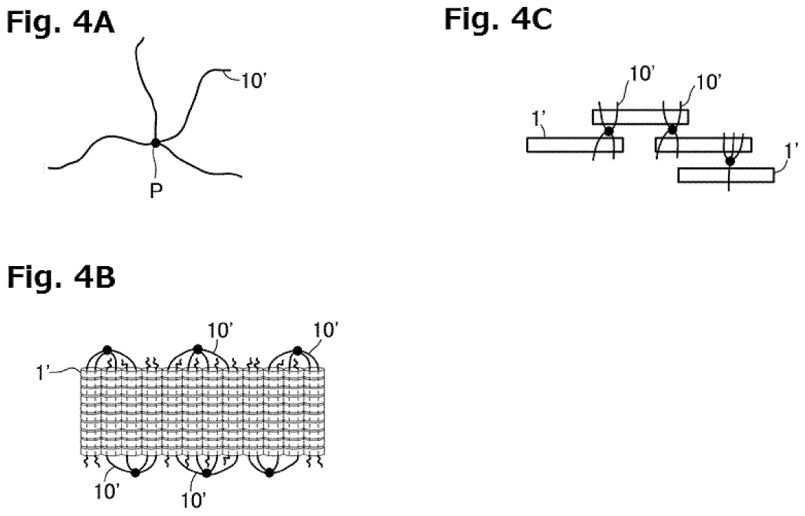

Next, the effect of the topology of the chain polymer 10 is described. When the chain polymer 10 is a single-chain PEO, as described above, structural bodies of various shapes are formed along with a change in molecular weight of the chain polymer. In contrast, when a PEO having a branching portion P illustrated in FIG. 4A is used as a chain polymer 10′, as illustrated in FIG. 4B, the branching point P suppresses the crystal growth of the cyclic molecule, and hence a sheet-shaped structure having a uniform thickness is formed. However, as illustrated in FIG. 4C, the chain polymer 10′ serves as a bridge to link the sheets. As described above, the topology of the chain polymer 10 may affect the behavior of the crystal of the structure.

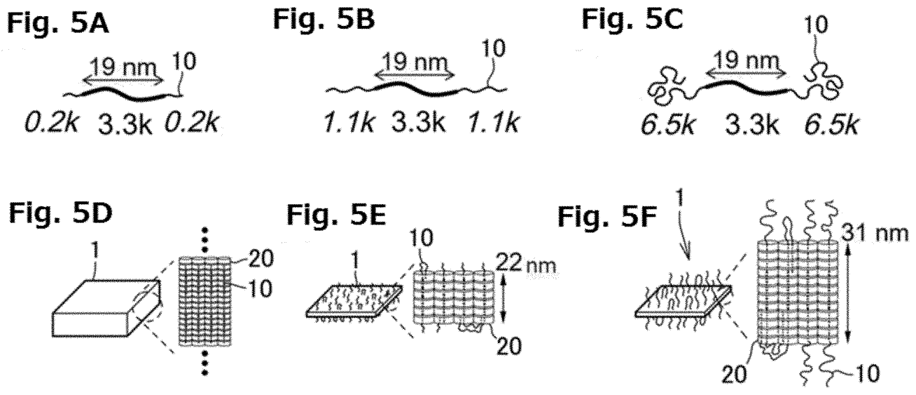

Next, the effect of a change in configuration of the polymer blocks of the chain polymer is described. For example, triblock polymers illustrated in FIG. 5A to FIG. 5C in each of which a central block is a PPO having a molecular weight of 3.3k (the symbol “k” means kilo, and the same is true for the following), and blocks on both sides are each a PEO having a molecular weight of 0.2k (FIG. 5A), 1.1k (FIG. 5B), or 6.5k (FIG. 5C) are each used as the chain polymer 10. In this case, block structural bodies illustrated in FIG. 5D to FIG. 5F are formed, respectively. In each case, with regard to the strength of an interaction with γ-CD, an interaction between γ-CD and the PPO is stronger than an interaction between γ-CD and the PEO, and hence γ-CD is localized to the center in the axial direction of the chain polymer 10. When the localization is utilized, in the case where the hydrophobic PPO block is arranged at the center of the chain polymer, the hydrophilic PEO blocks are arranged at both ends thereof, and the lengths of the PEO blocks are made sufficiently long, the PEO blocks at both the ends of the chain polymer 10 protrude from a column formed of a plurality of γ-CD molecules, and hence a single-layer sheet having a single thickness, that is, a sheet in which one pseudo-polyrotaxane and/or polyrotaxane molecule is arranged in its thickness direction can be produced.

Herein, the configuration of the structure according to at least one embodiment of the present invention is described.

As illustrated in FIG. 2, the structure 1 according to at least one embodiment of the present invention includes the plurality of pseudo-polyrotaxanes and/or polyrotaxanes 2. The pseudo-polyrotaxanes and/or polyrotaxanes 2 each include the chain polymer 10 included in the cavity of the cyclic molecule 20 in a skewered manner, and at least part of the plurality of pseudo-polyrotaxanes and/or polyrotaxanes 2 are arranged in series with each other. In addition, another part of the plurality of pseudo-polyrotaxanes and/or polyrotaxanes 2 are arranged in parallel with each other. The plurality of pseudo-polyrotaxanes and/or polyrotaxanes 2 are arranged in each of the thickness direction of the sheet of the structure 1 and two directions perpendicular to the thickness direction of the sheet. Specifically, in the figure, the two pseudo-polyrotaxanes and/or polyrotaxanes are arranged in series, and 17×10 pairs of the pseudo-polyrotaxanes and/or polyrotaxanes 2 are arranged in parallel.

The phrase “includes the plurality of pseudo-polyrotaxanes and/or polyrotaxanes” means that the structure includes the plurality of pseudo-polyrotaxanes, includes the plurality of polyrotaxanes, or includes the at least one pseudo-polyrotaxane and the at least one polyrotaxane.

The phrase “the plurality of pseudo-polyrotaxanes and/or polyrotaxanes are arranged in series with each other” means that the plurality of pseudo-polyrotaxanes and/or polyrotaxanes are arranged in a stacked manner in the axial directions of their cyclic molecules. One pseudo-polyrotaxane and/or polyrotaxane and the other pseudo-polyrotaxane and/or polyrotaxane that are arranged in series with each other are preferably in such a relationship that their axial directions substantially coincide with each other, and their cyclic molecules are arranged substantially in one row. However, as long as the cyclic molecules are arranged in a stacked manner in the axial directions of the cyclic molecules, the positions of the individual cyclic molecules may shift in a direction perpendicular to the axial directions to some extent.

The phrase “the plurality of pseudo-polyrotaxanes and/or polyrotaxanes are arranged in parallel with each other” means that the plurality of pseudo-polyrotaxanes and/or polyrotaxanes are arranged substantially parallel to each other. It is preferred that the axial directions of one pseudo-polyrotaxane and/or polyrotaxane and the other pseudo-polyrotaxane and/or polyrotaxane that are arranged in parallel with each other be substantially parallel to each other.

Although the size of the structure 1 is not particularly limited, dimensions in the a-axis, b-axis, and c-axis directions of the crystal of the structure 1 are typically of the order of nanometers (1 nm or more and less than 1,000 nm) or micrometers (1 μm or more and less than 1,000 μm). Pharmacokinetic behavior in a body varies depending on the particle size of the structure 1. For example, when the particle size is equal to or greater than 2 μm, the structure is taken in a liver cell, when the particle size is equal to or greater than 300 nm to 400 nm, the structure is captured and discharged by a macrophage, when the particle size is equal to or greater than 200 nm, the structure is treated in a spleen, and when the particle size is equal to or greater than 100 nm, the structure passes a space between vascular endothelial cells. Accordingly, the structure 1 can be designed by selecting the size of the structure 1 in accordance with purposes. Herein, the structure 1 whose dimension along at least one of the a-axis, the b-axis, or the c-axis is 1 μm or more is sometimes referred to as “microstructure.”

The structure 1 may take any one of the following shapes: a rod shape whose length in the c-axis direction is longer than its lengths in the a- and b-axis directions; a cube shape whose length in the c-axis direction is substantially equal to its lengths in the a- and b-axis directions; and a sheet shape whose length in the c-axis direction is shorter than its lengths in the a- and b-axis directions. In addition, when the structure 1 is of a sheet shape, the shape of the sheet in plan view may be a substantially square shape, a substantially rectangular shape, a rhombus, or a polygon (having 3, 4, 5, 6, or more sides). Further, the structure 1 may adopt any one of a tent shape, that is, a hollow pyramid shape, a polyhedral shape, a columnar shape (a prism shape or a cylindrical shape; including a solid or hollow shape), and a spherical shape (including a solid or hollow shape). FIG. 2 is an illustration of an example of the structure 1 of a sheet shape.

When the structure 1 is of a rod shape, its thickness (length in the c-axis direction) is preferably 100 nm or more, more preferably from 100 nm to 1,000 μm, still more preferably from 200 nm to 100 μm, and its length in each of the a-axis and b-axis directions is preferably 50 nm or more, more preferably from 50 nm to 100 μm, still more preferably from 100 nm to 10 μm.

When the structure 1 is of a cube shape, its thickness (length in the c-axis direction), and its length in each of the a-axis and b-axis directions are preferably 50 nm or more, more preferably from 50 nm to 1,000 μm, still more preferably from 100 nm to 100 μm.

When the structure 1 is of a sheet shape, its thickness (length in the c-axis direction) is preferably 50 nm or more, more preferably from 50 nm to 100 μm, still more preferably from 100 nm to 10 μm, and its length in each of the a-axis and b-axis directions is preferably 100 nm or more, more preferably from 100 nm to 1,000 μm, still more preferably from 200 nm to 100 μm.

The one chain polymer 10 may be housed in the plurality of cyclic molecules 20 of each of the pseudo-polyrotaxanes and/or polyrotaxanes 2, or the plurality of (e.g., two) chain polymers 10 may be housed therein.

Although the chain polymer 10 may be linear or may have a branch, the chain polymer 10 is preferably a single chain for controlling the configuration of the structure to prevent the linking of the structural bodies 1. In at least one exemplary embodiment, all of the plurality of chain polymers 10 for forming the respective pseudo-polyrotaxanes and/or polyrotaxanes 2 of the structure 1 are single chains. In at least one exemplary embodiment, part of the plurality of chain polymers 10 for forming the respective pseudo-polyrotaxanes and/or polyrotaxanes 2 of the structure 1 are single chains, and the other part thereof are branched chains.

When the pseudo-polyrotaxanes and/or polyrotaxanes 2 each have a capping group, examples of the capping group include, but not limited to, a dinitrophenyl group, a cyclodextrin, an adamantane group, a trityl group, fluorescein, and pyrene. A method of introducing the capping group into the chain polymer is known, and reference may be made to, for example, a method described in Harada et al., Nature, 1992, 356, 325-327.

The chain polymer 10 has a repeating structure derived from a monomer. A skeleton forming such repeating structure may be selected from the group consisting of: polyvinyl alcohol, polyvinylpyrrolidone, and poly(meth)acrylic acid; cellulose; modified celluloses, such as carboxymethylcellulose, hydroxyethylcellulose, and hydroxypropylcellulose; polyacrylamide, polyethylene oxide, polypropylene oxide, polytetrahydrofuran, polyvinyl acetal, polyvinyl methyl ether, polyamine, polyethyleneimine, casein, gelatin, and starch; polyolefins, such as polyethylene and polypropylene, a copolymer of an olefin, such as ethylene or propylene, with any other olefin, polyester, polyvinyl chloride, polystyrene, an acrylonitrile-styrene copolymer, an acrylonitrile-butadiene-styrene copolymer, polymethyl methacrylate, a (meth)acrylic acid ester copolymer, an acrylonitrile-methyl acrylate copolymer, polycarbonate, polyurethane, a vinyl chloride-vinyl acetate copolymer, polyvinyl butyral, polyisobutylene, polyaniline, polyamides, such as nylon, and polyimides; polydienes, such as polyisoprene and polybutadiene; and polysiloxanes, polysulfones, polyimines, polyacetic anhydrides, polyureas, polysulfides, polyphosphazenes, polyketones, polyphenylenes, and derivatives thereof.

The repeating structure of the chain polymer 10 is preferably, for example, at least one kind selected from the group consisting of polyethylene glycol, polyisoprene, polyisobutylene, polybutadiene, polypropylene glycol, polytetrahydrofuran, polydimethylsiloxane, polyethylene, polypropylene, polyvinyl alcohol, and polyvinyl methyl ether, more preferably at least one kind selected from the group consisting of polyethylene oxide and polypropylene oxide from the viewpoint of the interaction with the cyclic molecule 20.

The weight-average molecular weight of the chain polymer 10, which is not limited, is preferably from 200 to 200,000, more preferably from 200 to 50,000, still more preferably from 200 to 20,000.

When the structure 1 is of a rod shape, the weight-average molecular weight of the chain polymer 10 is preferably from 200 to 10,000, more preferably from 200 to 2,000, still more preferably from 200 to 1,000.

When the structure 1 is of a cube shape, the weight-average molecular weight of the chain polymer 10 is preferably from 1,000 to 20,000, more preferably from 2,000 to 10,000, still more preferably from 2,000 to 6,000.

When the structure 1 is of a sheet shape, the weight-average molecular weight of the chain polymer 10 is preferably from 2,000 to 200,000, more preferably from 4,000 to 100,000, still more preferably from 6,000 to 50,000.

In at least one exemplary embodiment, the chain polymer 10 is water-soluble, and examples thereof include at least one kind selected from the group consisting of polyethylene oxide (polyethylene glycol), polypropylene oxide (polypropylene glycol), polyvinyl alcohol, polyethyleneimine, polyacrylic acid, polymethacrylic acid, polyacrylamide, cellulose derivatives, such as hydroxypropylcellulose, and polyvinylpyrrolidone, more preferably at least one kind selected from the group consisting of polyethylene glycol and polypropylene glycol.

The weight-average molecular weight of the water-soluble chain polymer 10 is preferably from 200 to 200,000, more preferably from 200 to 50,000, still more preferably from 200 to 20,000.

The chain polymer 10 may be a polymer having a moiety formed by the polymerization of one kind of monomer, or formed only of such moiety, may be a polymer having a copolymer formed by the polymerization of two kinds of monomers, or formed only of such copolymer moiety, or may be a polymer having a terpolymer formed by the polymerization of three kinds of monomers, or formed only of such terpolymer moiety. Examples of those moieties include the examples described above as the skeleton for forming the repeating structure. In particular, examples of those moieties include, but not limited to, at least one kind selected from the group consisting of polyethylene glycol, polyisoprene, polyisobutylene, polybutadiene, polypropylene glycol, polytetrahydrofuran, polydimethylsiloxane, polyethylene, polypropylene, polyvinyl alcohol, and polyvinyl methyl ether.

The chain polymer 10 may be a block copolymer including two blocks. In addition, the chain polymer 10 may be a block copolymer including three blocks. When both the ends of the chain polymer 10 are housed in a column formed of the plurality of cyclic molecules 20, the adjacent cyclic molecules 20 of the adjacent pseudo-polyrotaxanes and/or polyrotaxanes can be arranged in series by a noncovalent bond interaction therebetween. In order that both the ends of the chain polymer 10 may be housed in the column formed of the plurality of cyclic molecules 20, it is preferred that no hydrophilic PEO blocks be arranged at both the ends of the chain polymer 10, or even when the hydrophilic PEO blocks are arranged, their lengths be 0.20 nm or less.

Preferred examples of the chain polymer 10 include, but not limited to, a single block polymer formed of polyethylene oxide (PEO), a diblock copolymer having a block formed of polyethylene oxide (PEO) and a block formed of polypropylene oxide (PPO), and a triblock copolymer having a block formed of polyethylene oxide (PEO), a block formed of polypropylene oxide (PPO), and a block formed of polyethylene oxide (PEO) in the stated order. The triblock copolymer formed of PEO-PPO-PEO is preferred because the PPO has hydrophobicity higher than that of the PEO, and hence the top of the PPO is more selectively included in the cyclic molecules in an aligned manner.

Each block of the block copolymer is preferably formed only of a repeating unit derived from one monomer, but may have a first spacer group between a certain repeating unit and the next repeating unit. In addition, a second spacer group that may be identical to or different from the first spacer group may be present between the adjacent blocks.

Examples of the first and/or second spacer group include, but not limited to, a linear or branched alkyl group having 1 to 20 carbon atoms, a linear or branched ether group having 1 to 20 carbon atoms, a linear or branched ester having 1 to 20 carbon atoms, and an aromatic group having 6 to 24 carbon atoms, such as a phenyl group.

When the cavity of the cyclic molecule 20 is held under the state of being penetrated by the chain polymer 10, any moiety of the chain polymer 10 may be included in the cyclic molecule 20. The chain polymer 10 formed by the polymerization of one kind of monomer may be included in the cyclic molecule. When the chain polymer is a block copolymer, one moiety out of the two moieties of the block copolymer may be included therein, or one moiety out of the three moieties of the block copolymer, in particular, the intermediate moiety out of the three moieties may be included therein.

When the chain polymer 10 is a block copolymer, the chain length of a moiety of the chain polymer 10 included in the cyclic molecule 20 is preferably longer than the thickness of the cyclic molecule 20. Herein, the thickness of the cyclic molecule 20 is schematically illustrated in FIG. 1A and FIG. 1B. The thickness of the cyclic molecule 20 refers to a thickness T along the central axis Ax of the cyclic molecule 20. The moiety of the chain polymer 10 included in the cyclic molecule 20 is a moiety formed of a polymer of one kind of monomer, one moiety out of the two moieties of the block copolymer, or one moiety out of the three moieties of the block copolymer.

The chain polymer 10 may have an ionizable group that is ionized in water or an aqueous solution. In at least one exemplary embodiment, the chain polymer 10 has the ionizable group at at least one terminal thereof or in the vicinity of the end. In at least one exemplary embodiment, the chain polymer 10 has the ionizable group at at least one terminal thereof. In at least one exemplary embodiment, the chain polymer 10 has the ionizable group at each of both terminals thereof.

Examples of the ionizable group include, but not limited to, at least one kind selected from the group consisting of a carboxyl group (which becomes —COO⁻ when ionized), an amino group (which becomes —NH³⁺ when ionized), a sulfo group, a phosphoric acid group, a trimethylamino chloride group, a triethylamino chloride group, a dimethylamino group, a diethylamino group, a methylamino group, an ethylamino group, a pyrrolidine group, a pyrrole group, an ethyleneimine group, a piperidine group, a pyridine group, a pyrylium ion group, a thiopyrylium ion group, a hexamethyleneimine group, an azide group, an imidazole group, a pyrazole group, an oxazole group, a thiazole group, an imidazoline group, a morpholine group, a thiazine group, a triazole group, a tetrazole group, a pyridazine group, a pyrimidine group, a pyrazine group, an indole group, a benzimidazole group, a purine group, a benzotriazole group, a quinoline group, a quinazoline group, a quinoxaline group, a pteridine group, a carbazole group, a porphyrin group, a chlorin group, a choline group, an adenine group, a guanine group, a cytosine group, a thymine group, a uracil group, a dissociated thiol group, a dissociated hydroxy group, an azido group, a pyridine group, carbamic acid, guanidine, sulfene, urea, thiourea, peroxy acid, derivatives thereof, and analogs thereof.

The ionizable group is preferably at least one kind selected from the group consisting of a carboxyl group, an amino group, a sulfo group, a phosphoric acid group, a trimethylamino chloride group, and a dimethylamino group, more preferably at least one kind selected from the group consisting of a carboxyl group, an amino group, a trimethylamino chloride group, and a dimethylamino group.

The chain polymer 10 may have a non-ionizable group instead of the ionizable group. A conventionally known method may be used for the introduction of the ionizable group or the non-ionizable group into the chain polymer.

Examples of the non-ionizable group include, but not limited to, at least one kind selected from the group consisting of: an isopropyl group, a sec-butyl group, a tert-butyl group, a neopentyl group, an isopentyl group, a sec-pentyl group, a 3-pentyl group, a tert-pentyl group, a cyclopentyl group, a pentene group, a hexyl group, a hexene group, a heptyl group, a heptene group, an octyl group, an octene group, a nonyl group, a nonene group, a decyl group, a decene group, an undecyl group, an undecene group, a dodecyl group, a dodecene group, a tridecyl group, a tridecene group, a tetradecyl group, a tetradecene group, a pentadecyl group, a pentadecene group, a hexadecyl group, a hexadecene group, a heptadecyl group, a heptadecene group, an octadecyl group, an octadecene group, a nonadecyl group, a nonadecene group, an eicosyl group, an eicosene group, a tetracosyl group, a tetracosene group, a triacontyl group, and a triacontene group, and isomers thereof; and a 4-tert-butylbenzenesulfonyl group, a 2-mesitylenesulfonyl group, a methanesulfonyl group, a 2-nitrobenzenesulfonyl group, a 4-nitrobenzenesulfonyl group, a pentafluorobenzenesulfonyl group, a 2,4,6-triisopropylbenzenesulfonyl group, a p-toluenesulfonyl group, a hydroxy group that is not ionized, a heptafluorobutyroyl group, a pivaloyl group, a perfluorobenzoyl group, an amino group (—NH₂) that is not ionized, a carboxyl group (—COOH) that is not ionized, and an isovaleryl group.

The non-ionizable group is preferably at least one kind selected from the group consisting of a hydroxy group that is not ionized, a heptafluorobutyroyl group, a perfluorobenzoyl group, and an isovaleryl group, more preferably at least one kind selected from the group consisting of a perfluorobenzoyl group and an isovaleryl group.

Examples of the cyclic molecule 20 include, but not limited to, a cyclodextrin (e.g., α-cyclodextrin, β-cyclodextrin, or γ-cyclodextrin), a crown ether, a pillararene, a calixarene, a cyclophane, a cucurbituril, and derivatives thereof. Examples of the derivatives include, but not limited to, methylated α-cyclodextrin, methylated β-cyclodextrin, methylated γ-cyclodextrin, hydroxypropylated α-cyclodextrin, hydroxypropylated β-cyclodextrin, and hydroxypropylated γ-cyclodextrin.

The structure 1 may include one kind of the cyclic molecule 20, or may include two or more kinds of the cyclic molecules 20. For example, when the structure 1 includes two or more kinds of the cyclic molecules 20, the structure may include a first cyclic molecule that is one of the compounds listed above and a second cyclic molecule that is one of the compounds listed above and is different from the first cyclic molecule.

Herein, the ratio of the cyclic molecule 20 in the pseudo-polyrotaxane and/or polyrotaxane 2 is referred to as “inclusion ratio.”

The term “specified inclusion ratio” refers to an inclusion ratio arithmetically specified from the chain polymer 10 and the cyclic molecule 20 in the pseudo-polyrotaxane and/or the polyrotaxane 2, and is specifically specified from the length of the chain polymer 10 and the thickness T of the cyclic molecule 20.

A method of calculating the specified inclusion ratio is described with reference to a case in which polyethylene glycol is used as the chain polymer 10 and α-cyclodextrin is used as the cyclic molecule.

It has been known from molecular model calculation that the thickness of the two repeating units of the polyethylene glycol is the same as that of α-cyclodextrin. Therefore, a specified inclusion ratio when a molar ratio between α-cyclodextrin and the repeating units of the polyethylene glycol is set to 1:2 is defined as 100%.

The inclusion ratio, that is, the ratio of the cyclic molecule 24 in the pseudo-polyrotaxane and/or polyrotaxane 2 may be determined by small-angle X-ray scattering (SAXS) measurement of a dispersion of the obtained structure.

The inclusion ratio of the pseudo-polyrotaxane and/or the polyrotaxane 2 is preferably from 1% to 100%, more preferably from 5% to 100%, still more preferably from 10% to 100%, most preferably from 20% to 100% when the specified inclusion ratio is set to 100%.

In at least one exemplary embodiment, to secure a high occupancy of the cyclic molecules 20 in the structure, the chain polymer 10 is included in the cavities of the three or more cyclic molecules 20 per one chain polymer in a skewered manner.

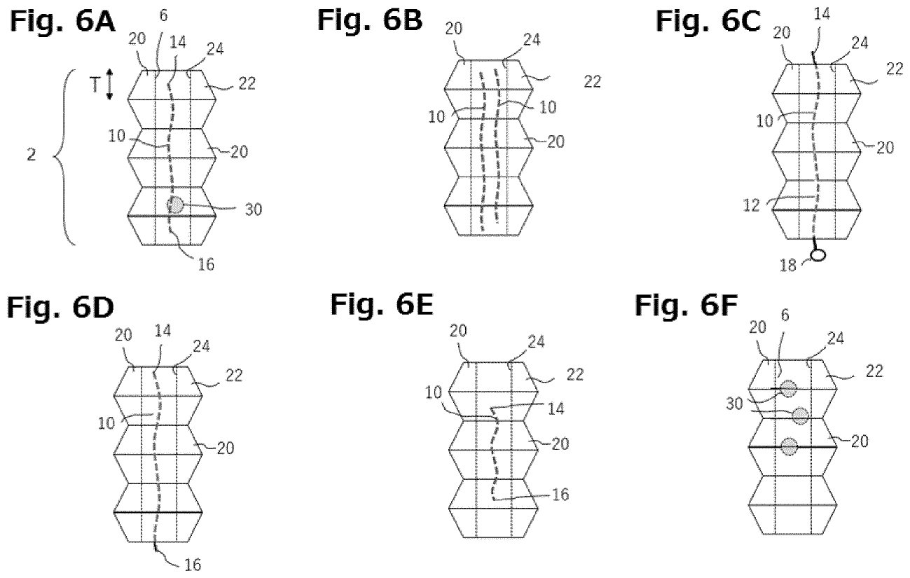

Returning to FIG. 2, as long as the structure 1 according to at least one embodiment of the present invention can be formed, the structure 1 may further include a component except the pseudo-polyrotaxanes and/or polyrotaxanes 2, that is, an additional substance 30 (see FIG. 6F) except the above-mentioned cyclic molecule 20 and the above-mentioned chain polymer 10 included in the cavity 24 of the cyclic molecule 20 in a skewered manner. Such substance 30 is, for example, a first substance that is included in the cyclic molecule 20, a second substance that is not included in the cyclic molecule 20, or both of the substances.

Examples of the first substance include a drug, a fluorescent substance, and a chromogenic enzyme.

Examples of the drug include, but not limited to, an antitumor drug, an antihypertensive, an antihypotensive, an antipsychotic, an analgesic, an antidepressant, an antimanic drug, an antianxiety, a sedative, an anti-dementia drug, a hypnotic, an anti-tantrum drug, an antiasthmatic, an anesthetic, an antiarrhythmic, an antiarthritic, an antispasmodic, an ACE inhibitor, a decongestant, an antibiotic, an antianginal, a diuretic, an antiparkinsonism drug, a bronchodilator, an antidiuretic, a diuretic, a hypolipidemic, an immunosuppressive, an immunomodulator, an antiemetic, an anti-infective, an antineoplastic, an antifungal, an antiviral, an antidiabetic, an antiallergic, an antifebrile, an antipodagric, an antihistamine, an antipruritic, a bone regulator, a cardiovascular drug, a hypocholesterolemic drug, an antimalarial, an antitussive, an expectorant, a mucolytic, a dopaminergic drug, a gastrointestinal drug, a muscle relaxant, a neuromuscular blocker, a parasympathomimetic drug, a stimulant, an anorectic, a thyroid drug or an antithyroid drug, a hormone, an antimigraine drug, an anti-obesity drug, an anti-inflammatory drug, a drug for renal diseases, a urologic agent, an ophthalmic drug, a dermatologic drug, and a dental and oral drug.

A preferred drug in terms of ease of inclusion in the cyclic molecule 20 is, for example, a steroid (in particular, a compound having the basic skeleton of cyclopentahydrophenanthrene), an anthracycline, or a fluorine-containing nucleoside.

Examples of the drug include, but not limited to: hydrocortisone, spironolactone, clofibrate, naproxen, adenine arabinoside, adenosine, ibuprofen, hydrochlorothiazide, acetylsalicylic acid, methyl salicylate, adamantane, azobenzene, anthracene, pyrene, polyphenylene vinylene, polyaniline, rhodamine, Nile red, ethenzamide, prednisolone acetate, rebamipide, salbutamol, flurbiprofen, piroxicam, ketoprofen, Timoptol, dorzolamide, doxorubicin, isopropyl unoprostone, diphenylhydramine, hydroxyzine, cetirizine, chlorpheniramine, epinastine, levocabastine, levofloxacin, latanoprost, bimatoprost, tafluprost, timolol, a basic fibroblast growth factor (bFGF), carboplatin, cisplatin, tegafur, nedaplatin, paclitaxel, pirarubicin, 5-fluorouracil, bleomycin, mitomycin, salicylic acid, dexamethasone, cytarabine, nimustine, dacarbazine, docetaxel, amphotericin, pancratistatin, phenytoin, diazepam, daunorubicin, astemizole, beclomethasone, betamethasone, bendazac, bromazepam, celecoxib, chlordiazepoxide, clobazam, clonazepam, coenzyme Q10, cortisone, curcumin, cyproterone acetate, fluocinolone acetate, flurazepam, flutamide, indomethacin, ketotifen, loratadine, lorazepam, medazepam, meloxicam, natamycin, nimesulide, nimetazepam, nitrazepam, nystatin, prednisolone, progesterone, risperidone, sildenafil, telmisartan, testosterone, triamcinolone, felbinac, suprarenal extract/heparinoid, loxoprofen sodium hydrate, diclofenac sodium, fluocinolone, loteprednol, difluprednate, rimexolone, fluorometholone, metasulfobenzoate sodium, betamethasone sodium phosphate, dexamethasone sodium phosphate, cefmenoxime, ofloxacin, chloramphenicol, and donepezil; and a salt of any of the compounds acceptable as a pharmaceutical (e.g., salbutamol sulfate, doxorubicin hydrochloride, nimustine hydrochloride, dorzolamide hydrochloride, nylhydramine hydrochloride, hydroxyzine hydrochloride, cetirizine hydrochloride, chlorpheniramine maleate, epinastine hydrochloride, levocabastine hydrochloride, timolol maleate, beclomethasone hydrochloride, cefmenoxime hydrochloride, or donepezil hydrochloride).

Examples of the fluorescent substance include, but not limited to, rhodamine, Nile red, poly-L-lysine-fluorecein isothiocyanate (FITC), uranine, coumarine, Cy2, Cy3, and Cy5.

Examples of the chromogenic enzyme include, but not limited to, horseradish peroxidase (HRP), alkaline phosphatase, β-galactosidase, glucose oxidase, and luciferase.

The second substance is, for example, a polymer that is not included in the cyclic molecule 20.

Examples of the polymer that is not included in the cyclic molecule 20 include, but not limited to: polymers, such as polystyrene, polyvinylpyridine, polypyridine, polyphenylene, polyacrylamide, polyacrylic acid, polymethacrylic acid, polyvinyl alcohol, polyamide, polyester, polyimide, polybenzoxazole, polyvinyl chloride, polyolefins (e.g., polyethylene and polypropylene), polysilane, and polysiloxane; biopolymers, such as DNA and a protein formed of 50 or more amino acids bonded to each other; biomolecules, such as a peptide formed of less than 50 amino acids bonded to each other; inorganic nanomaterials, such as silica nanoparticles, titanium oxide nanoparticles, and silicon nanoparticles; carbon materials, such as fullerene, carbon nanotubes, graphene, graphite, and carbon quantum dots; metal/inorganic nanoparticles, such as gold nanoparticles, perovskite quantum dots, CdSeS/ZnS quantum dots, iron oxide nanoparticles, and nanodiamond; and cell adhesion molecules, such as fibronectin, collagen, cadherin, integrin, laminin, fibrinogen, and polylysine.

The substance 30 may be bonded to the chain polymer 10, may be bonded to the cyclic molecule 20, may be held in a space 4 between the plurality of pseudo-polyrotaxanes and/or polyrotaxanes 2 (i.e., when a plurality of, in particular, 2, 3, or 4 columns that are columnar structures formed of the pseudo-polyrotaxanes and/or polyrotaxanes are present, a space therebetween) (illustrated in FIG. 2), may be housed in the cavity 24 defined and formed by the one cyclic molecule 20 (illustrated in FIG. 1B), or may be housed in the space 6 defined and formed by the plurality of cyclic molecules 20 (illustrated in FIG. 1B and FIG. 2). When the substance 30 is bonded to the chain polymer 10, the substance is preferably bonded to each of both the ends, or one end, of the chain polymer 10, or the vicinity of the end, but may be bonded to another site of the chain polymer 10. In addition, to house the substance 30 in the space 6 defined and formed by the plurality of cyclic molecules 20, the chain polymer 10 is not preferably housed therein, but a molecule except the substance 30 typified by the chain polymer 10 may be housed therein.

The size of the space 4 between the plurality of pseudo-polyrotaxanes and/or polyrotaxanes 2, the size of the cavity 24 defined and formed by the one cyclic molecule 20, and the size of the space 6 defined and formed by the plurality of cyclic molecules 20 may be appropriately changed by changing, for example, the length of the chain polymer 10, the hydrophilicity and hydrophobicity of the chain polymer 10, and the kind of the cyclic molecule 20. Accordingly, the size of the space 4, the size of the cavity 24, and/or the size of the space 6 only needs to be appropriately changed in accordance with the size of the substance 30 that is wished to be housed.

The structure 1 according to at least one embodiment of the present invention is thicker than the related-art single-layer nanosheet, and hence a large amount of the substance 30, such as a drug, can be taken in the one structure. Accordingly, the structure 1 according to at least one embodiment of the present invention can function as a vehicle for the drug to enable the lengthening of the sustained release time of the drug.

Further, the structure 1 according to at least one embodiment of the present invention includes a molecule having high biosafety, and is hence suitable for utilization in a living organism.

In addition, when the structure 1 according to at least one embodiment of the present invention is produced by using the short chain polymer 10 as a raw material, the production can be performed more rapidly, and energy and cost can be reduced.

As illustrated in FIG. 6A to FIG. 6F, the chain polymer 10 and the cyclic molecule 20 in the structure 1 may adopt various configurations to the extent that the structure 1 can exhibit an intended function as long as the configuration of the structure 1 as an assembly is maintained.

For example, in FIG. 6A, the plurality of (six in the figure) cyclic molecules 20 form a column, and the one chain polymer 10 is housed in the space 6 formed by the continuation of the cavities 24 of the cyclic molecules 20. The one chain polymer 10 extends over the cavities 24 of the plurality of cyclic molecules 20, but both the ends of the chain polymer 10 do not reach both ends of the column formed of the plurality of cyclic molecules 20 but are housed in the space 6. The column formed of the plurality of cyclic molecules 20 may also be called a laminate formed of the plurality of cyclic molecules 20, that is, a stack. The substance 30 may be housed in the cavities 24 of the cyclic molecules 20 or the space 6 formed by the plurality of cyclic molecules 20, or may not be housed therein.

In FIG. 6B, the two chain polymers 10 are housed in the space 6, and the two chain polymers 10 extend across the cavities 24 of the plurality of cyclic molecules 20. Both the ends of each of the chain polymers 10 reach both the ends of the column formed of the plurality of cyclic molecules 20, and hence the total height of the plurality of cyclic molecules 20 for forming the one pseudo-polyrotaxane and/or polyrotaxane 2 substantially corresponds to the total length of the polymer 10.

In FIG. 6C, both the ends of the chain polymer 10 slightly protrude from the cyclic molecules 20 to the outside. One end of the main body 12 of the chain polymer 10 has the modifying group 18.

In FIG. 6D, the one end 14 of the chain polymer 10 is housed in the space 6, and the other end 16 thereof slightly protrudes from the cyclic molecules 20 to the outside.

In FIG. 6E, the one chain polymer 10 is housed in the space 6, and such chain polymer 10 extends to the cavities 24 of the four cyclic molecules 20 but does not extend to the cavities 24 of the top and bottom cyclic molecules 20. That is, the length of the chain polymer 10 is short, and is a half or less of the length of the space 6 (i.e., the total height of the plurality of cyclic molecules 20).

In FIG. 6F, only the column formed of the plurality of cyclic molecules 20 is present, and the chain polymer 10 is absent. In this embodiment, the substance 30 is housed in the space 6 formed by the plurality of cyclic molecules 20.

In at least one exemplary embodiment, all of the columns each formed of the plurality of cyclic molecules 20 in the structure 1 each include the chain polymer 10. In at least one exemplary embodiment, part of the columns each formed of the plurality of cyclic molecules 20 in the structure 1 each include the chain polymer 10, and the other part of the columns are each free of the chain polymer 10.

As described above, the size of the space 4, the size of the cavity 24, and/or the size of the space 6 may be appropriately designed and adjusted in accordance with the size of the substance 30 that is wished to be housed. In addition, the occupancy of the size of the space 4, the size of the cavity 24, and/or the size of the space 6 in the structure 1 may be appropriately designed and adjusted. Accordingly, for example, when the substance 30 is a drug, the drug is housed in a desired amount in the space 4, cavity 24, and/or space 6 of the structure 1, and hence the structure 1 can be caused to function as a drug-encapsulated body or a drug release-controlling carrier.

The amount of the substance 30 in the structure 1 can be measured by absorbance measurement. For example, the substance concentration-absorbance calibration curve of a solution, which is obtained by dissolving the substance 30 at a known concentration in a solvent, at a predetermined wavelength is measured in advance. A predetermined amount of the structure 1 is dissolved in the same solvent, and the absorbance of the solution is measured, followed by the determination of the absorbance value thereof at the predetermined wavelength. The concentration of the substance is calculated from the determined absorbance value and the calibration curve, and the amount of the substance 30 in the structure 1 is calculated. In at least one embodiment, the amount of the substance 30 in the structure 1 is 0.0001 mass % or more, and may be more specifically, but not limited to, from 0.001 mass % to 11 mass %. In the case of the substance 30 in the structure 1 or the substance 30 to be encapsulated in the structure 1, the substance 30 comprehends the substance 30 housed in the space 6 defined and formed by the cyclic molecules 20, the substance 30 that is not included in the cyclic molecule 20 but present between the plurality of cyclic molecules 20, and the substance 30 that is not included in the cyclic molecule 20 but adheres not to a space between the plurality of cyclic molecules 20 but to the outer surface of the structure 1.

The preferred structure 1 according to at least one embodiment of the present invention is excellent in adhesive property with a solid base material. The solid base material may be a nonbiological solid base material, or may be a biological object. Of the base materials, a base material of a plate shape is referred to as “substrate.” Examples of the nonbiological solid base material include a solid base material formed of an inorganic material and a solid base material formed of an organic material. Examples of the solid base material formed of the inorganic material include, but not limited to, glass, a metal, a metal oxide, silicon, quartz, and zirconia. Examples of the solid base material formed of the organic material include, but not limited to, a synthetic resin and a biopolymer.

The biological object is, for example, part of a biological tissue.

In addition, the structure 1 may be widely used as a encapsulated body or release-controlling carrier for the substance 30 not only in medicine but also in fields including bioimaging, food, cosmetics, and industrial chemistry. The structure 1 according to at least one embodiment of the present invention is applied in a wide variety of fields as, for example, a drug delivery material, a reagent for bioimaging, a masking material for a food molecule (in particular, a volatile molecule, such as an odorant), a hair care material, a coating material, a paint, an adhesive, a wound-healing material, an artificial biological alternative material, a packaging material, a rubber material, an oral care material, such as a mouthwash, a base for a supplement, a functional beverage, or a material for controlling the aggregation of cells, algae, and the like.

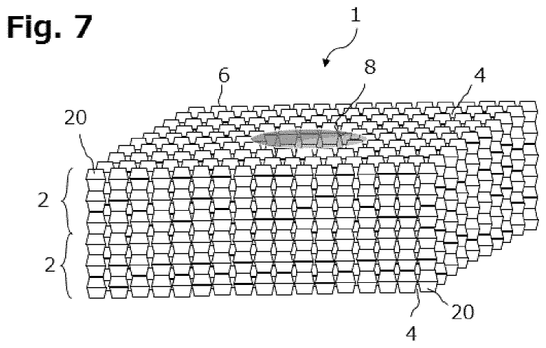

As illustrated in FIG. 7, in at least one specific embodiment, the structure 1 includes the basic structure of the embodiment illustrated in FIG. 2, and further has one or a plurality of through-holes 8 (one through-hole in the figure) penetrating the structure 1. Although the sizes of the respective through-holes 8 are not limited, the through-holes each preferably occupy a space larger than a volume occupied by the one pseudo-polyrotaxane and/or polyrotaxane 2. The one pseudo-polyrotaxane and/or polyrotaxane 2, which may be any one of the pseudo-polyrotaxanes and/or polyrotaxanes 2 in the structure 1, is, for example, the pseudo-polyrotaxane and/or polyrotaxane having the largest volume. The through-holes 8 can be formed by: causing the structure 1 according to at least one embodiment of the present invention to adhere to a substrate; and washing the resultant with water or an aqueous solution at a certain temperature for a certain time period. The washing temperature, which is not limited, is, for example, from 5° C. to 45° C., preferably from 15° C. to 30° C. The washing time, which is not limited, is, for example, from 10 seconds to 60 seconds.

Next, a method of producing the structure 1 is described.

Production methods I and II for the structure according to at least one embodiment of the present invention are provided.

### <Production Method I>

The production method I includes the steps of:

(a) preparing the chain polymer 10;

(b) preparing the cyclic molecule 20; and

(c) mixing the chain polymer 10 and the cyclic molecule 20 in water or an aqueous solution. Such production method can provide a structure including a plurality of pseudo-polyrotaxanes and/or polyrotaxanes each including the chain polymer 10 included in the cavity of the cyclic molecule 20 in a skewered manner, in which at least part of the plurality of pseudo-polyrotaxanes and/or polyrotaxanes are arranged in series with each other.

In the step (c), the plurality of pseudo-polyrotaxanes and/or polyrotaxanes each including the chain polymer having both ends housed in a column formed of the plurality of cyclic molecules interact with each other, and hence at least part of the plurality of pseudo-polyrotaxanes and/or polyrotaxanes are arranged in series with each other.

The “chain polymer 10” and the “cyclic molecule 20” are as described above.

<Step (a)>

The step (a) is a step of preparing the chain polymer 10.

Herein, the chain polymer 10 may be purchased on the market, or may be prepared. When the “chain polymer 10” is prepared, the chain polymer 10 may be obtained by, for example, methods described in the following documents 1 to 4.
Document 1: Hillmyer, M. A. et al., Macromolecules 1996, 29(22) 6994-7002. Document 2: Ding, J. F. et al., Eur Polym J 1991, 27(9), 901-905. Document 3: Allegaier, J. et al., Macromolecules 2007, 40(3), 518-525. Document 4: Malik, M. I. et al., Eur Po. ym J 2009, 45(3), 899-910.
   <Step (b)>

The step (b) is a step of preparing the cyclic molecule 20.

In this step, the cyclic molecule may be purchased on the market, or may be prepared. When a derivative is to be prepared, the derivative may be obtained by, for example, a method described in Document 5: Khan, A. R. et al., Chem Rev 1998, 98(5), 1977-1996 or the like.

The step (b) only needs to be provided before the step (c). That is, the step (b) does not need to be provided after the step (a), and the steps (a) and (b) may be performed separately from each other.

<Step (c)>

The step (c) is a step of mixing the chain polymer 10 and the cyclic molecule 20 in water or an aqueous solution. The water or the aqueous solution is not particularly limited as long as the water or the aqueous solution serves as a solvent capable of dissolving at least one of the cyclic molecule 20 or the chain polymer 10.

Specific examples of the water or the aqueous solution to be used in the step (c) may include, but not limited to, pure water, an aqueous solution of an alcohol, an aqueous solution of an acid, an aqueous solution of an alkali, a buffer, a culture medium, and blood plasma.

The above-mentioned structure can be obtained by the production method including the steps (a) to (c).

The above-mentioned production method may include a step other than the steps (a) to (c). Examples of the step other than the steps (a) to (c) may include, but not limited to: a step of preparing the above-mentioned “chain polymer 10,” which is provided before the step (a); a step of purifying the structure, which is provided after the step (d); and the inclusion of the cyclic molecule and the first substance and the synthesis of pseudo-polyrotaxanes or polyrotaxanes, which may be provided before the step (a).

In addition, when the structure has the above-mentioned substance 30, the production method according to at least one embodiment of the present invention may include a step for the introduction of the substance 30 into the structure 1.

To introduce the substance 30 into the structure 1, first, the chain polymer 10 and the substance 30, such as a drug, are mixed in water or an aqueous solution to be dissolved or dispersed therein. When the substance 30 contains a substance that is hardly water-soluble, an ultrasonic wave may be applied to the mixed solution. After that, the cyclic molecule 20 is loaded into the mixed solution, and the mixture is shaken until the molecule is sufficiently dissolved or dispersed therein. Thus, the structure 1 having taken therein the substance 30 is obtained. After that, a suspension containing the structure 1 is centrifuged so that the structure 1 may be precipitated. The precipitate is subjected to washing with an aqueous solution or water containing the cyclic molecule 20 and centrifugation several times, and the supernatant is removed. Thus, the structure 1 having taken therein the substance 30 is obtained.

Further, it is desired to further include, after the step (c), a step of modifying part of the pseudo-polyrotaxanes of the obtained structure.

The modifying step may be a step of introducing a first substituent into the chain polymer 10, for example, at an end of the chain polymer 10. As long as the structure is obtained, the first substituent may be a capping group having such a capping action as to prevent dissociation of the cyclic molecule 20, or may have any other action. The first substituent may have any combination of those actions, and may exhibit all the actions.

Examples of a group having the capping action may include, but not limited to, an adamantane group, a neopentyl group, an isopentyl group, a sec-pentyl group, a 3-pentyl group, a tert-pentyl group, a cyclopentyl group, a pentene group, a hexyl group, a hexene group, a heptyl group, a heptene group, an octyl group, an octene group, a nonyl group, a nonene group, a decyl group, a decene group, an undecyl group, an undecene group, a dodecyl group, a dodecene group, a tridecyl group, a tridecene group, a tetradecyl group, a tetradecene group, a pentadecyl group, a pentadecene group, a hexadecyl group, a hexadecene group, a heptadecyl group, a heptadecene group, an octadecyl group, an octadecene group, a nonadecyl group, a nonadecene group, an eicosyl group, an eicosene group, a henicosyl group, a henicosene group, a tetracosyl group, a tetracosene group, a triacontyl group, and a triacontene group, and isomers thereof.

For example, a group derived from folic acid, biotin, fluorescein, an oligopeptide, such as RGD or GRGDS, or a monoclonal antibody, such as rituximab, bevacizumab, tocilizumab, or infliximab, may be introduced as a group having the other action, for example, the action of an ionizable group. For example, when a group derived from folic acid is to be introduced, the introduction may be performed by subjecting the isolated sheet to be obtained and folic acid to a reaction in the presence of a condensing agent, such as 4-(4,6-dimethoxy-1,3,5-triazin-2-yl)-4-methylmorpholinium chloride (DMT/MM), N,N′-dicyclohexylcarbodiimide (DCC), 1-(3-dimethylaminopropyl)-3-ethylcarbodiimide (EDC), benzotriazol-1-yloxy-trisdimethylaminophosphonium salt (BOP), (benzotriazol-1-yloxy)tripyrrolizidinophosphonium hexafluorophosphate (PyBOP), or O-(7-azabenzotriazol-1-yl)-N,N,N′,N′-tetramethyluronium hexafluorophosphate (HATU).

The modifying step may be a step of introducing a second substituent into the cyclic molecule 20 as long as the structure is obtained.

### <Production Method II>

The production method II includes the steps of:

(a′) preparing the chain polymers 10;

(b) preparing the cyclic molecules 20;

(c′) mixing the chain polymers 10 and the cyclic molecules 20 in water or an aqueous solution to provide pseudo-polyrotaxanes;

(d) introducing substituents into both terminals of at least part of the chain polymers 10 to provide chain polymers 10;

(e) introducing capping groups into both terminals of at least part of the chain polymers 10 of the pseudo-polyrotaxanes and/or the chain polymers 10; and

(f) mixing the resultant pseudo-polyrotaxanes and/or polyrotaxanes in water or an aqueous solution. Such production method can provide a structure including a plurality of pseudo-polyrotaxanes and/or polyrotaxanes each including the chain polymer 10 included in the cavity of the cyclic molecule 20 in a skewered manner, in which at least part of the plurality of pseudo-polyrotaxanes and/or polyrotaxanes are arranged in series with each other.

In the step (f), the plurality of pseudo-polyrotaxanes and/or polyrotaxanes each including the chain polymer having both ends housed in a column formed of the plurality of cyclic molecules interact with each other, and hence at least part of the plurality of pseudo-polyrotaxanes and/or polyrotaxanes are arranged in series with each other.

Herein, in the step (a′), the “chain polymer 10” described in the above-mentioned step (a) may be used. The step (b) is the same as the above-mentioned “step (b).”

As in the above-mentioned step (c), the step (c′) is a step of mixing the chain polymers 10 and the cyclic molecules 20 in the water or the aqueous solution, and is a step of obtaining the pseudo-polyrotaxanes through the mixing. As described in the step (c), the water or the aqueous solution is not particularly limited as long as the water or the aqueous solution is a solvent in which at least one of the cyclic molecule 20 or the chain polymer 10 is soluble.

Specific examples of the water or the aqueous solution to be used in the step (c) may include, but not limited to, pure water, an alcohol aqueous solution, an acid aqueous solution, an alkali aqueous solution, a buffer, a culture solution, and plasma.

The step (d) is a step of introducing the substituents into both the terminals of at least part of the chain polymers 10 to provide the chain polymers 10.

As a nonlimitative example of the introduction of the substituents described above, a carboxylic acid can be introduced by an oxidation reaction including using hypochlorous acid and 2,2,6,6-tetramethylpiperidine 1-oxyl. An amino group can be introduced by a coupling reaction including using 1,1′-carbonyldiimidazole and ethylenediamine A sulfo group can be introduced by causing 1,3-propanesultone to react with the chain polymer 10.

Other nonlimitative examples of the introduction of the substituents include, but not limited to, a condensation reaction, such as esterification and amidation, using a condensation agent, such as 4-(4,6-dimethoxy-1,3,5-triazin-2-yl)-4-methylmorpholinium chloride (DMT/MM), N,N′-dicyclohexylcarbodiimide (DCC), 1-(3-dimethylaminopropyl)-3-ethylcarbodiimide (EDC), benzotriazol-1-yloxy-trisdimethylaminophosphonium salt (BOP), (benzotriazol-1-yloxy)tripyrrolidinophosphonium hexafluorophosphate (PyBOP), or O-(7-dibenzotriazol-1-yl)-N,N,N′,N′-tetramethyluronium hexafluorophosphate (HATU), a nucleophilic substitution reaction, and an addition reaction.

The step (e) is a step of introducing the so-called capping groups, and is a step of introducing the capping groups to reduce the desorption rate of the cyclic molecule 20. A conventionally known approach may be used for the step, and the step may be, for example, a step described in Harada et al., Nature, 1992, 356, 325-327. In addition, examples of the capping groups may include capping groups that can be used for conventionally known polyrotaxanes. Examples thereof may include capping groups described in M. Okada et al., J Polym. Sci. A: Polym. Chem, 2000, 38, 4839-4849.

According to at least one embodiment of the present invention, there is provided the structure having a larger thickness in the axial direction of the chain polymer (axial direction of the cyclic molecule having included therein the chain polymer). In addition, the method of producing such structure is advantageous in that energy, cost, or a production time is reduced.

The present invention also comprehends the following embodiments.

Item 1. A structure including a plurality of pseudo-polyrotaxanes and/or polyrotaxanes each including a chain polymer included in a cavity or cavities of one or more cyclic molecules in a skewered manner, at least part of the plurality of pseudo-polyrotaxanes and/or polyrotaxanes being arranged in series with each other.

Item 2. The structure according to Item 1, wherein the chain polymer is a single chain.

Item 3. The structure according to Item 1 or 2, wherein the chain polymer is included in the cavities of the three or more cyclic molecules per one chain polymer in a skewered manner.

Item 4. The structure according to any one of Items 1 to 3, wherein at least part of the plurality of pseudo-polyrotaxanes and/or polyrotaxanes are arranged in parallel with each other.

Item 5. The structure according to any one of Items 1 to 4, further including a substance that is bonded to the chain polymer, is bonded to the cyclic molecule, or is held in one of a space between the plurality of pseudo-polyrotaxanes and/or polyrotaxanes, the cavity defined and formed by the one cyclic molecule, and a space defined and formed by the plurality of cyclic molecules.

Item 6. The structure according to Item 5, wherein the substance is at least one selected from the group consisting of a drug, a fluorescent substance, and a chromogenic enzyme.

Item 7. The structure according to any one of Items 1 to 6, wherein the chain polymer has a length of from 1 nm to 2,000 nm.

Item 8. The structure according to any one of Items 1 to 7, wherein the chain polymer includes a moiety formed of one of polyethylene glycol (PEG) or polypropylene glycol (PPG).

Item 9. The structure according to any one of Items 1 to 8, wherein the cyclic molecule is at least one selected from the group consisting of a cyclodextrin, a crown ether, a pillararene, a calixarene, a cyclophane, and derivatives thereof.

Item 10. The structure according to any one of Items 1 to 9, wherein the structure has arranged therein at least one through-hole penetrating the structure and occupying a space larger than a volume occupied by the one pseudo-polyrotaxane and/or polyrotaxane.

Item 11. The structure according to any one of Items 1 to 10, wherein the structure is any one of the following (a) to (c):

(a) a rod-shaped structure in which the chain polymer has a weight-average molecular weight of from 200 to 10,000;

(b) a cube-shaped structure in which the chain polymer has a weight-average molecular weight of from 1,000 to 20,000; and

(c) a sheet-shaped structure in which the chain polymer has a weight-average molecular weight of from 2,000 to 200,000.

Item 12. A method of producing a structure including a plurality of pseudo-polyrotaxanes and/or polyrotaxanes each including a chain polymer included in a cavity or cavities of one or more cyclic molecules in a skewered manner, the method including causing the plurality of pseudo-polyrotaxanes and/or polyrotaxanes each including the chain polymer having both ends housed in a column formed of the plurality of cyclic molecules to interact with each other to arrange at least part of the plurality of pseudo-polyrotaxanes and/or polyrotaxanes in series with each other.

The present invention is more specifically described by way of Examples, but the present invention is not limited thereto.

### EXAMPLES

### Example 1 Formation of Structure Using γ-CD

γ-CD was obtained from CycloChem Co., Ltd. A chain polymer PEO 2k (the number represents a weight-average molecular weight) was purchased from each of Sigma-Aldrich Co. LLC and Wako Pure Chemical Industries, Ltd.

The preparation of a pseudo-polyrotaxane (PPR) is as described below. γ-CD was dissolved in deionized water (having a pH of about 7) at 23° C.±1° C. The γ-CD concentration of the solution was set to 0.12 gmL⁻¹. 0.25 g of the PEO 2k was added to 8.4 mL of the γ-CD aqueous solution. The mixed solution was stirred with a vortex for 1 minute. Next, the solution was placed on a shaker and aged. The precipitate and the liquid layer were separated from each other within 1 day, and hence composite formation was substantially completed. The sample is named PPR_EO 400.

A sample for a scanning electron microscope (SEM) was prepared by immersing a silicon oxide substrate in an aqueous dispersion of the pseudo-polyrotaxane. SEM observation was performed with a JEOL JSM-7800F apparatus. In addition, in-situ crystal growth induced by the complex formation was recorded with a Nikon ECLIPSE Ts2R phase-contrast microscope including a Nikon DS-Fi3 camera.

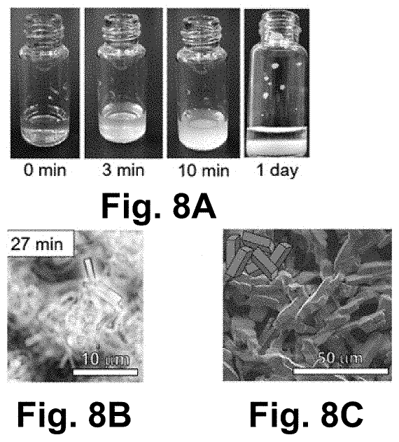

A photograph at the time of observation with naked eyes in the formation of the PPR_EO 400 is shown in FIG. 8A. The solution gradually became opaque along with the formation of the structure. In addition, a photograph obtained by the observation of the PPR_EO 400 with an optical microscope and a photograph obtained by the observation thereof with the SEM are shown in FIG. 8B and FIG. 8C, respectively. It was recognized that a rod-shaped structure was formed.

### Example 2 Control of Shape of Structure Based on Length of Chain Polymer

γ-CD was obtained from CycloChem Co., Ltd Chain polymers PEO 400 (the number represents a weight-average molecular weight), PEO 2k, PEO 4k, PEO 6k, and PEO 20k were purchased from each of Sigma-Aldrich Co. LLC and Wako Pure Chemical Industries, Ltd.

Methods of preparing pseudo-polyrotaxanes (PPRs) are the same as that of Example 1. The produced samples were named PPR_EO 400, PPR_EO 2k, PPR_EO 4k, PPR_EO 6k, and PPR_EO 20k, respectively.

SEM observation was performed under the same experimental conditions as those of Example 1. WAXS measurement was performed as follows: an aqueous dispersion of each of the pseudo-polyrotaxanes was injected into a glass capillary (WJM-Glass/Muller GmbH Boro-Silicate capillary: φ=2.0 mm×length=80 mm); the sample was dried at room temperature for 1 day; and then the measurement was performed by using particles adhering to a glass wall as targets.

The same apparatus as that of Example 1 was used in the SEM observation. The WAXS experiment was performed with a Rigaku NANOPIX apparatus including a HyPix-3000 detector. A distance from each of the samples to the detector was calibrated with respect to the diffraction peak of silver behenate.

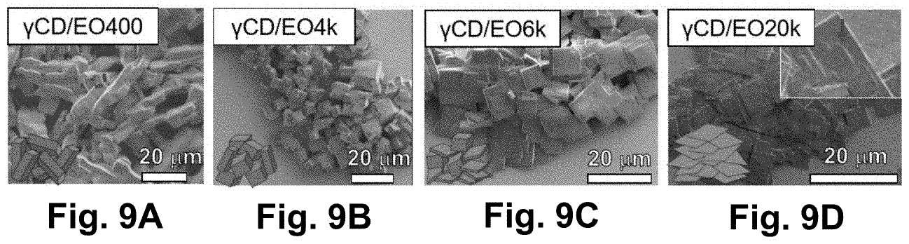

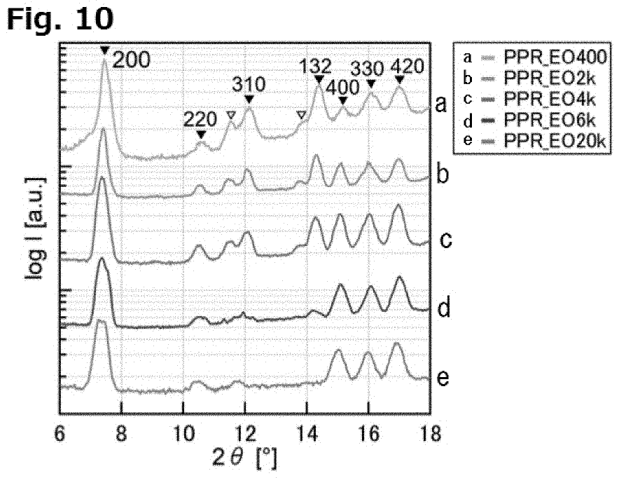

Photographs obtained by the SEM observation of the PPR_EO 400, the PPR_EO 4k, the PPR_EO 6k, and the PPR_EO 20k are shown in FIG. 9A, FIG. 9B, FIG. 9C and FIG. 9D, respectively. It was recognized that the PPR_EO 400 formed a rod-shaped structure, the PPR_EO 4k formed a cube-shaped structure, and the PPR_EO 6k and the PPR_EO 20k formed sheet-shaped structural bodies. The analysis of the crystal structures of the samples by WAXS showed that tetragons were formed in all the samples. Crystal faces corresponding to the peaks of the samples were shown in FIG. 10.

### Example 3 Influence of Hydrophilicity/Hydrophobicity of Chain Polymer

γ-CD was obtained from CycloChem Co., Ltd Chain polymers PPO 400 (the number represents a weight-average molecular weight), PPO 2k, and PPO 4k were purchased from each of Sigma-Aldrich Co. LLC and Wako Pure Chemical Industries, Ltd.

Methods of preparing pseudo-polyrotaxanes (PPRs) are the same as that of Example 1. The produced samples were named PPR_PO 400, PPR_PO 2k, and PPR_PO 4k, respectively.

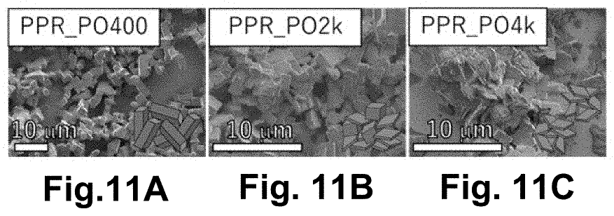

SEM observation was performed under the same experimental conditions as those of Example 1. The respective SEM images were shown in FIG. 11. A rod-shaped structure changed to a sheet-shaped structure along with an increase in molecular weight of the chain polymer. However, an amorphous structure was formed in the PPR_PO 4k. As the molecular weight of the linear polymer increases, the linear polymer needs to fold. However, when the linear polymer is hydrophobic, its hydrophobic aggregation occurs, and hence an amorphous structure is formed. Meanwhile, when the linear polymer is hydrophilic, no hydrophobic aggregation occurs, and hence a structure of an ordered shape is formed.

### Example 4 Formation of Ultrathin Structure when Triblock Copolymer is Used as Chain Polymer

γ-CD was obtained from CycloChem Co., Ltd Chain triblock polymers EO₄PO₅₆EO₄ (the subscripts each represent the number of units), EO₂₅PO₅₆EO₂₅, and EO₁₄₇PO₅₆EO₁₄₇ were purchased from Sigma-Aldrich Co. LLC.

Methods of preparing pseudo-polyrotaxanes (PPRs) are the same as that of Example 1. The produced samples were named PPR_EO₄PO₅₆EO₄, PPR_EO₂₅PO₅₆EO₂₅, and PPR_EO₁₄₇PO₅₆EO₁₄₇, respectively.

Samples for atomic force microscope (AFM) observation and SEM observation were each prepared by immersing a silicon oxide substrate in an aqueous dispersion of each of the pseudo-polyrotaxanes. An AFM experiment was performed with a Bruker Nano Multimode 8 apparatus operated in a tapping mode under ambient conditions. An antimony-doped silicon cantilever tip (Bruker RTESPA-300) having a resonance frequency of about 300 Hz and a spring constant of 40 Nm⁻¹ was used. SAXS measurement was performed by injecting the aqueous dispersion of the pseudo-polyrotaxane into a glass capillary (WJM-Glass/Muller GmbH Boro-Silicate capillary: φ=2.0 mm×length=80 mm). A WAXS experiment was performed under the same conditions as those of Example 2.

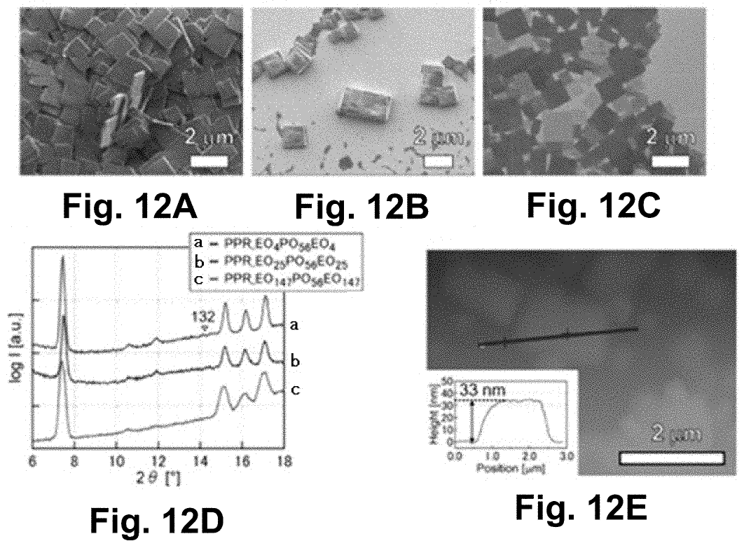

The respective SEM images are shown in FIG. 12A, FIG. 12B, and FIG. 12C. In the PPR_EO₄PO₅₆EO₄ and the PPR_EO₂₅PO₅₆EO₂₅, relatively thick sheet-shaped structural bodies were formed. In the PPR_EO₁₄₇PO₅₆EO₁₄₇, however, an ultrathin structure was formed. It was found from a WAXS profile (FIG. 12D) that a tetragonal crystal was formed. The thickness of the PPR_EO₁₄₇PO₅₆EO₁₄₇ was measured with the AFM. As a result, the thickness was found to be 33 nm (FIG. 12E).

### Example 5 Production of Nanosheet Having Through-Hole

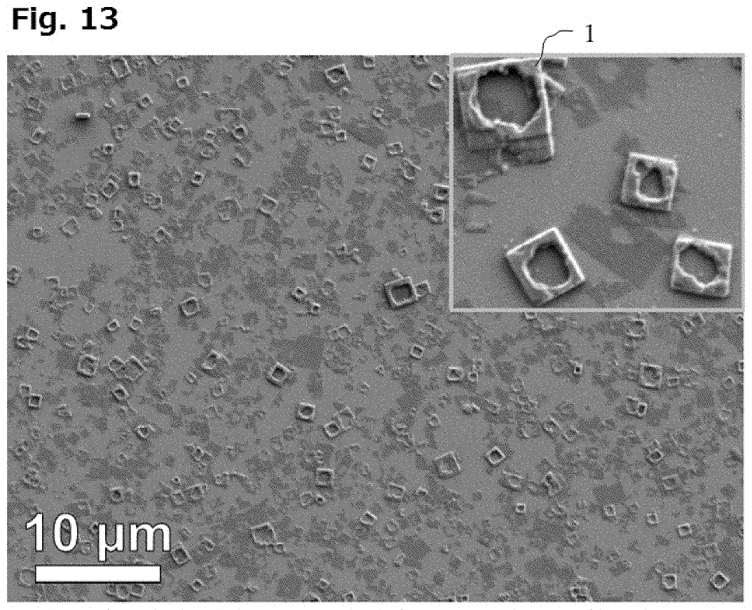

The PPR_EO₁₄₇PO₅₆EO₁₄₇ produced in Example 4 was caused to adhere to a silicon oxide substrate, and then the resultant was washed with water for 20 seconds, followed by the performance of SEM observation (FIG. 13). It was recognized that a nanosheet structure having a through-hole was formed.

### Example 6 Formation of Microstructure Using α-CD

α-CD was purchased from Nihon Shokuhin Kako Co., Ltd. A chain polymer PEO 400 (the number represents a weight-average molecular weight) was purchased from Sigma-Aldrich Co. LLC.

The preparation of a pseudo-polyrotaxane (PPR) is as described below. α-CD was dissolved in deionized water (having a pH of about 7) at 23° C.±1° C. The α-CD concentration of the solution was set to 0.12 gmL⁻¹. 0.25 g of the PEO 400 was added to 8.4 mL of the α-CD aqueous solution. The mixed solution was stirred with a vortex for 1 minute. Next, the solution was placed on a shaker and aged. The precipitate and the liquid layer were separated from each other within 1 day, and hence composite formation was substantially completed.

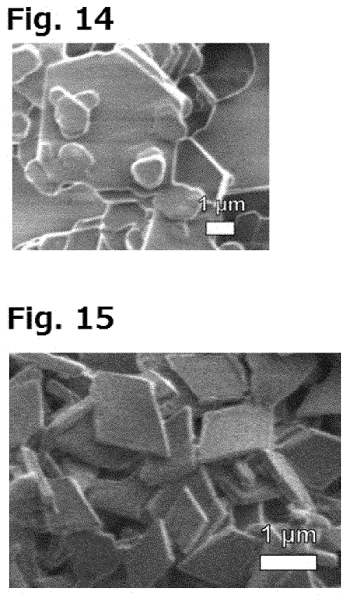

SEM observation was performed by the same approach as that of Example 1 (FIG. 14). It was recognized that a microstructure of a hexagonal prism shape was formed.

### Example 7 Formation of Microstructure Using β-CD

β-CD was purchased from FUJIFILM Wako Pure Chemical Corporation. Poly(propylene glycol) bis(2-aminopropyl ether) 2k (the number represents a weight-average molecular weight, NH₂PPO 2k) was purchased from Sigma-Aldrich Co. LLC.

The preparation of a pseudo-polyrotaxane (PPR) is as described below. β-CD was dissolved in deionized water (having a pH of about 7) at 23° C.±1° C. The β-CD concentration of the solution was set to 18 mgmL⁻¹. 1 mg of the NH₂PPO 2k was added to 1.0 mL of the β-CD aqueous solution. The mixed solution was stirred with a vortex for 1 minute. Next, the solution was placed on a shaker and aged. The precipitate and the liquid layer were separated from each other in 1 week, and hence composite formation was substantially completed. The sample was named β-CD/NH₂PPO 2k.

SEM observation was performed by the same approach as that of Example 1 (FIG. 15). It was recognized that a microstructure of a rhomboid shape was formed.

### Example 8 Formation of Microstructure Using Axial Molecule Except PEO and PPO

β-CD was purchased from FUJIFILM Wako Pure Chemical Corporation. γ-CD was obtained from CycloChem Co., Ltd. Polytetrahydrofuran 1.4k (the number represents a weight-average molecular weight, PTHF 1.4k) was purchased from each of Wako Pure Chemical Industries, Ltd. and Sigma-Aldrich Co. LLC. Silanol-terminated polydimethylsiloxane 700-1,500 (PDMS 1.1k) and PDMS 2k-3.5k (PDMS 2.75k) were purchased from AZmax Co., Ltd. A PDMS 1,000-PEO 2,100 diblock copolymer (PDMS 1k-PEO 2.1k) was purchased from Polymer Source Inc.

The preparation of a PPR formed of β-CD is as described below. β-CD was dissolved in deionized water (having a pH of about 7) at 23° C.±1° C. The β-CD concentration of the solution was set to 18 mgmL⁻¹. 1 mg of the PDMS 1.1k and NH₂PPO 2k were added to 1.0 mL of the β-CD aqueous solution. The mixed solution was stirred with a vortex for 1 minute. Next, the solution was placed on a shaker and aged. The precipitate and the liquid layer were separated from each other in 1 week, and hence composite formation was substantially completed. The sample was named β-CD/PDMS 1.1k.

The preparation of a PPR formed of γ-CD is as described below. γ-CD was dissolved in deionized water (having a pH of about 7) at 23° C.±1° C. The γ-CD concentration of the solution was set to 0.12 gmL⁻¹. 0.25 g of each of the PDMS 2.75k, the PDMS 1k-PEO 2.1k, and the PTHF 1.4k was added to 8.4 mL of the γ-CD aqueous solution. The mixed solution was stirred with a vortex for 1 minute. Next, the solution was placed on a shaker and aged. The precipitate and the liquid layer were separated from each other within 1 day, and hence composite formation was substantially completed. The samples are named γ-CD/PDMS 2.75k, γ-CD/PDMS 1k-PEO 2.1k, and γ-CD/PTHF 1.4k, respectively.

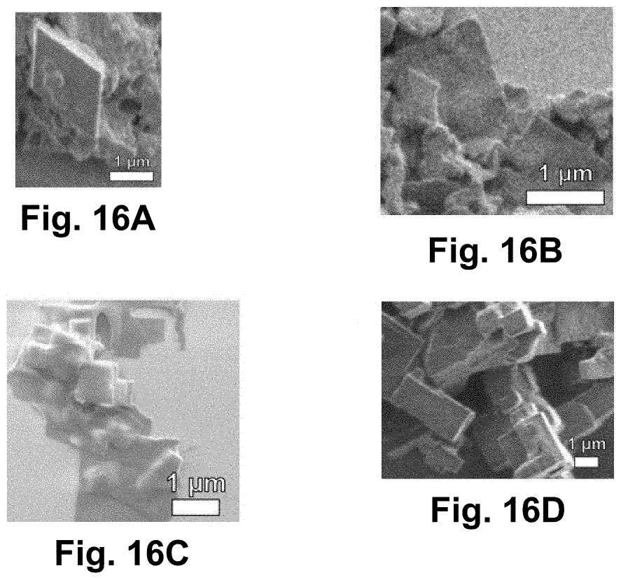

SEM observation was performed by the same approach as that of Example 1. The SEM images of the β-CD/PDMS 1.1k, the γ-CD/PDMS 2.75k, the γ-CD/PDMS 1k-PEO 2.1k, and the γ-CD/PTHF 1.4k are shown in FIG. 16A to FIG. 16D, respectively. It was recognized that in the β-CD/PDMS 1.1k, a rhomboid microstructure was formed, and in the γ-CD/PDMS 2.75k, the γ-CD/PDMS 1k-PEO 2.1k, and the γ-CD/PTHF 1.4k, tetragonal microstructural bodies were formed.

### Example 9 Formation of Tent-Shaped Structure

γ-CD was obtained from CycloChem Co., Ltd. Perfluorohexanoic acid (C₅F₁₁—COOH) was purchased from Tokyo Chemical Industry Co., Ltd.

The preparation of a PPR formed of γ-CD is as described below. γ-CD was dissolved in deionized water (having a pH of about 7) at 23° C.±1° C. The γ-CD concentration of the solution was set to 0.12 gmL⁻¹. 0.14 g of C₅F₁₁—COOH was added to 1.25 mL of the γ-CD aqueous solution. The mixed solution was stirred with a vortex for 1 minute. Next, the solution was placed on a shaker and aged. The precipitate and the liquid layer were separated from each other within 1 day, and hence composite formation was substantially completed. The sample is named γ-CD/C₅F₁₁—COOH.

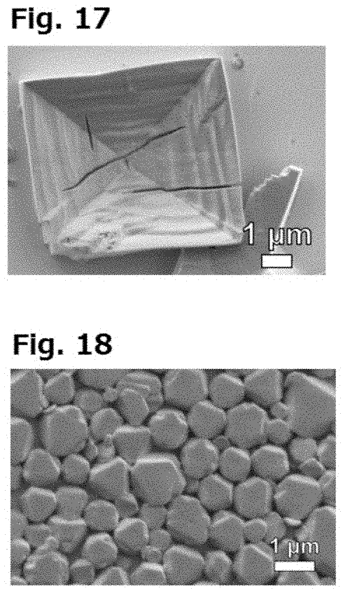

SEM observation was performed by the same approach as that of Example 1. The SEM image of the γ-CD/C₅F₁₁—COOH is shown in FIG. 17. It was recognized that a tent-shaped structure (facing down) was formed.

### Example 10 Formation of Polyhedral Microstructure

α-CD was purchased from Nihon Shokuhin Kako Co., Ltd. Sodium hexadecyl sulfonate (C₁₆H₃₃SO₃Na) was purchased from Tokyo Chemical Industry Co., Ltd.

The preparation of a pseudo-polyrotaxane (PPR) is as described below. α-CD was dissolved in deionized water (having a pH of about 7) at 23° C.±1° C. The α-CD concentration of the solution was set to 0.12 gmL⁻¹. 0.12 g of C₁₆H₃₃SO₃Na was added to 2.5 mL of the α-CD aqueous solution. The mixed solution was stirred with a vortex for 1 minute. Next, the solution was placed on a shaker and aged. The precipitate and the liquid layer were separated from each other within 1 day, and hence composite formation was substantially completed. The sample is named α-CD/C₁₆H₃₃SO₃Na.

SEM observation was performed by the same approach as that of Example 1. The SEM image of the α-CD/C₁₆F₃₃SO₃Na is shown in FIG. 18. It was recognized that a polyhedral microstructure was formed.

### Example 11 Formation of Sheet-Shaped Microstructure

α-CD was purchased from Nihon Shokuhin Kako Co., Ltd. Sodium 1-nonanesulfonate (C₉H₁₉SO₃Na) was purchased from Tokyo Chemical Industry Co., Ltd.

The preparation of a pseudo-polyrotaxane (PPR) is as described below. α-CD was dissolved in deionized water (having a pH of about 7) at 23° C.±1° C. The α-CD concentration of the solution was set to 0.12 gmL⁻¹. 86 mg of C₉H₁₉SO₃Na was added to 2.5 mL of the α-CD aqueous solution. The mixed solution was stirred with a vortex for 1 minute. Next, the solution was placed on a shaker and aged. The precipitate and the liquid layer were separated from each other within 1 day, and hence composite formation was substantially completed. The sample is named α-CD/C₉H₁₉SO₃Na.

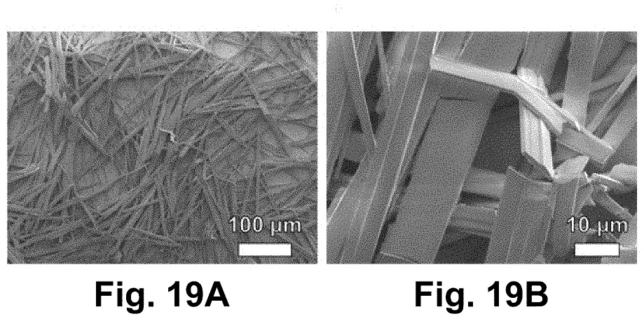

A sample for structural analysis was prepared by the same approach as that of Example 1. The results of the SEM observation of the α-CD/C₉H₁₉SO₃Na are shown in FIG. 19A and FIG. 19B (FIG. 19A corresponds to a low magnification and FIG. 19B corresponds to a high magnification). It was recognized that a sheet-shaped microstructure was formed.

### Example 12 Formation of Hollow Spherical Structure

α-CD was purchased from Nihon Shokuhin Kako Co., Ltd. A chain triblock polymer EO₃₇PO₅₆EO₃₇ (the subscripts each represent the number of units) was purchased from Sigma-Aldrich Co. LLC.

The preparation of a pseudo-polyrotaxane (PPR) is as described below. α-CD was dissolved in deionized water (having a pH of about 7) at 23° C.±1° C. The α-CD concentration of the solution was set to 0.12 gmL⁻¹. 0.25 g of the EO₃₇PO₅₆EO₃₇ was added to 8.4 mL of the α-CD aqueous solution. The mixed solution was stirred with a vortex for 1 minute. Next, the solution was placed on a shaker and aged. The precipitate and the liquid layer were separated from each other within 1 day, and hence composite formation was substantially completed. The sample is named α-CD/EO₃₇PO₅₆EO₃₇.

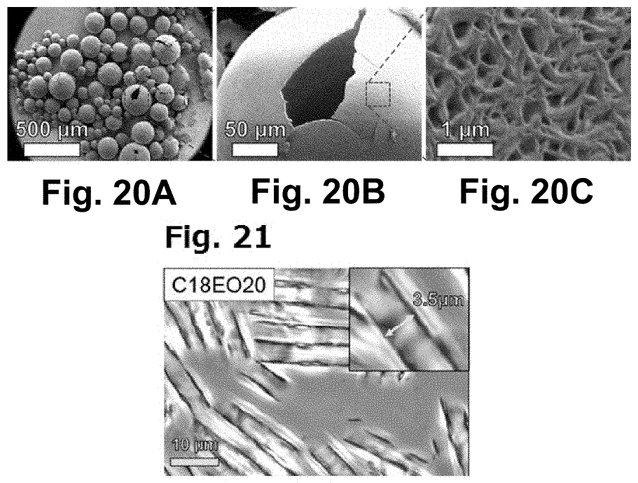

A sample for structural analysis was prepared by the same approach as that of Example 1. The results of the SEM observation of the α-CD/EO₃₇PO₅₆EO₃₇ are shown in FIG. 20A, FIG. 20B, and FIG. 20C (FIG. 20A corresponds to a low magnification, FIG. 20B corresponds to a medium magnification, and FIG. 20C corresponds to a high magnification). It was recognized that a hollow spherical structure was formed.

### Example 13 Formation of Hollow Columnar Structure

β-CD was purchased from FUJIFILM Wako Pure Chemical Corporation. Polyethylene glycol monooleyl ether (n=20, C₁₈EO₂₀) was purchased from Tokyo Chemical Industry Co., Ltd.

The preparation of a PPR is as described below. β-CD was dissolved in deionized water (having a pH of about 7) at 23° C.±1° C. The β-CD concentration of the solution was set to 18 mgmL⁻¹. 81 mg of the C₁₈EO₂₀ was added to 5.0 mL of the β-CD aqueous solution. The mixed solution was stirred with a vortex for 1 minute. Next, the solution was placed on a shaker and aged. The precipitate and the liquid layer were separated from each other within 1 day, and hence composite formation was substantially completed. The sample was named β-CD/C₁₈EO₂₀.

Optical microscope observation was performed by the same approach as that of Example 1. The result of the optical microscope observation of the β-CD/C₁₈EO₂₀ is shown in FIG. 21. It was recognized that a hollow columnar structure was formed.

### Example 14 Encapsulation of Anticancer Drug in Microstructure

γ-CD was obtained from CycloChem Co., Ltd. Polyethylene oxide 4.6k (hereinafter “PEO 4.6k,” weight-average molecular weight: 4.6k) was purchased from Sigma-Aldrich Co. LLC. Doxorubicin hydrochloride was purchased from FUJIFILM Wako Pure Chemical Corporation.

The preparation of an anticancer drug-encapsulated microstructure is as described below. 120 mg of γ-CD and 45 mg of doxorubicin were dissolved in 1 mL of deionized water (having a pH of about 7) at 23° C.±1° C. The γ-CD concentration of the solution was set to 120 mg/mL, and the doxorubicin hydrochloride concentration thereof was set to 4.5 mg/mL. 30 mg of the PEO 4.6k was added to 1 mL of the prepared aqueous solution of γ-CD and doxorubicin hydrochloride. The mixed solution was stirred with a vortex for 1 minute. Next, the solution was placed on a shaker and aged. The precipitate and the liquid layer were separated from each other within 1 day, and hence composite formation was substantially completed. The sample is named Dox-PPR_EO 4.6k.

Microscope observation was performed with Nikon ECLIPSE Ts2R including a Nikon DS-Fi3 camera. In addition, in fluorescence observation, light having a wavelength of 470 nm was used as an excitation light.

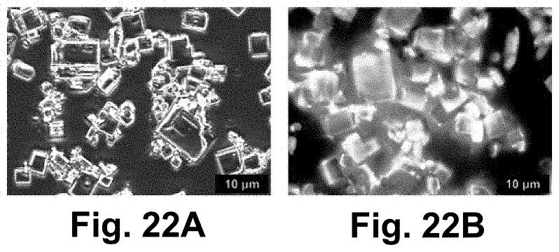

The results of the phase-contrast microscope observation and fluorescence microscope observation of the Dox-PPR_EO 4.6k are shown in FIG. 22A and FIG. 22B, respectively. In the phase-contrast microscope observation, it was able to be recognized that a microstructure was formed. In addition, it was recognized by the fluorescence microscope observation that the intensity of fluorescence derived from doxorubicin increased in a region where the microstructure was present, and hence doxorubicin was significantly taken in the microstructure.

### Example 15 Adhesion Property of Sheet-Shaped Nanostructure

β-CD was purchased from FUJIFILM Wako Pure Chemical Corporation. EO₇₅PO₃₀EO₇₅ (the subscripts each represent the number of units) was purchased from Sigma-Aldrich Co. LLC.

The preparation of a sheet-shaped nanostructure is as described below. 18 mg of β-CD was dissolved in 1 mL of deionized water (having a pH of about 7) at 23° C.±1° C. 4 mg of EO₇₅PO₃₀EO₇₅ was added to 1 mL of the prepared β-CD aqueous solution. The mixed solution was stirred with a vortex for 1 minute. Next, the solution was placed on a shaker and aged. The precipitate and the liquid layer were separated from each other in 1 week, and hence composite formation was substantially completed. The sample is named β-CD/EO₇₅PO₃₀EO₇₅.

An aqueous dispersion of the produced β-CD/EO₇₅PO₃₀EO₇₅ that was a sheet-shaped nanostructure was dropped on 3 kinds of solid base materials (pig skin, hair, and a contact lens), and moisture was wiped off with filter paper, followed by the performance of SEM observation. The resultant samples are named Skin_β-CD/EO₇₅PO₃₀EO₇₅, hair_β-CD/EO₇₅PO₃₀EO₇₅, and lens_β-CD/EO₇₅PO₃₀EO₇₅, respectively. In addition, the PPR_EO₁₄₇PO₅₆EO₁₄₇ produced in Example 4 was dropped on a pig eye, and the resultant was washed with a saline solution having a physiological saline concentration (0.9 wt %). After that, moisture was wiped off with filter paper, and then the remainder was observed with an optical microscope. The resultant sample is defined as eye_PPR_EO₁₄₇PO₅₆EO₁₄₇.

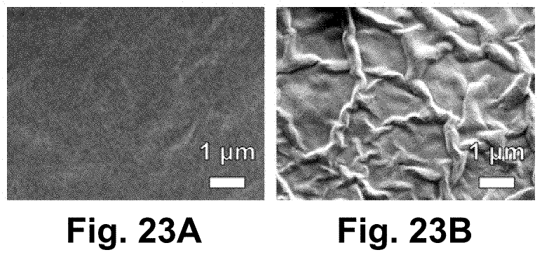

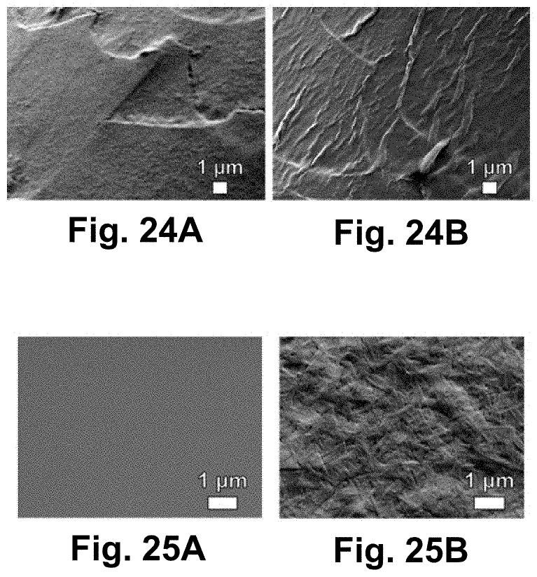

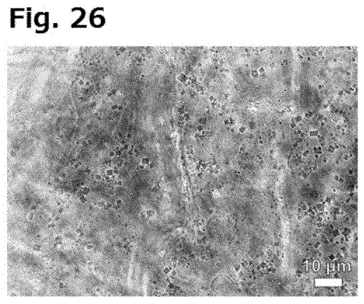

The SEM images of the Skin_β-CD/EO₇₅PO₃₀EO₇₅, the hair_β-CD/EO₇₅PO₃₀EO₇₅, and the lens_β-CD/EO₇₅PO₃₀EO₇₅ were shown in FIG. 23A and FIG. 23B, FIG. 24A and FIG. 24B, and FIG. 25A and FIG. 25B, respectively, and a photograph obtained by the optical microscope observation of the eye_PPR_EO₁₄₇PO₅₆EO₁₄₇ was shown in FIG. 26. In each case, a manner in which the sheet-shaped nanostructure adhered to the base material was able to be recognized.

### Example 16 UV-Cutting Function of Sheet-Shaped Nanostructure

β-CD was purchased from FUJIFILM Wako Pure Chemical Corporation. EO₇₅PO₃₀EO₇₅ (the subscripts each represent the number of units) was purchased from Sigma-Aldrich Co. LLC.

A sheet-shaped nanostructure was prepared by the same approach as that of Example 15. The sample is named β-CD/EO₇₅PO₃₀EO₇₅.

The UV-cutting function of a dispersed water of the β-CD/EO₇₅PO₃₀EO₇₅ was evaluated with a UV-visible spectrophotometer (UV3150, Shimadzu Corporation). The wavelength of incident light was set to from 250 nm to 800 nm, and the absorbance of the dispersed water was measured.

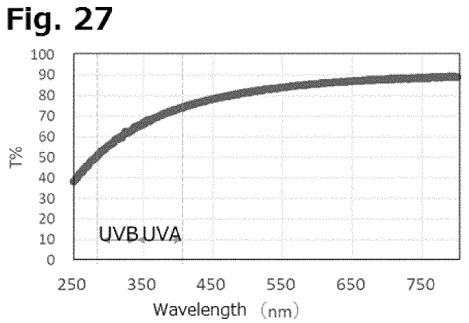

The results of the measurement are shown in FIG. 27. It was found that the dispersed water had a low transmittance in the wavelength regions of UVA (from 315 nm to 400 nm) and UVB (from 290 nm to 320 nm), and transmitted the light in a visible wavelength region (from 400 nm to 700 nm).

### Example 17 Gas Barrier Function of Coating Formed of Sheet-Shaped Nanostructure

β-CD was purchased from FUJIFILM Wako Pure Chemical Corporation. EO₇₅PO₃₀EO₇₅ (the subscripts each represent the number of units) was purchased from Sigma-Aldrich Co. LLC.

A sheet-shaped nanostructure was prepared by the same approach as that of Example 15. The sample is named β-CD/EO₇₅PO₃₀EO₇₅.

5 mL of a dispersed water of the β-CD/EO₇₅PO₃₀EO₇₅ and 20 mL thereof were each passed through a porous membrane (PMMA-millipore: TYPE JCWP 10.0 μm, hydrophilic, pore diameter: about 10 μm) to bond the β-CD/EO₇₅PO₃₀EO₇₅ to the membrane. After that, the membranes were naturally dried at room temperature. Those samples are named pore-β-CD/EO₇₅PO₃₀EO₇₅ (5 mL) and pore-β-CD/EO₇₅PO₃₀EO₇₅ (20 mL).

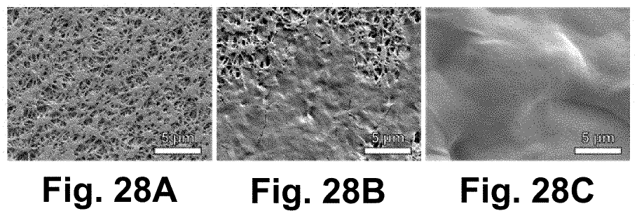

The SEM images of the porous membrane, the pore-β-CD/EO₇₅PO₃₀EO₇₅ (5 mL), and the pore-β-CD/EO₇₅PO₃₀EO₇₅ (20 mL) are shown in FIG. 28A, FIG. 28B, and FIG. 28C, respectively. It was able to be recognized that the porous membrane was perforated with many pores, and in the pore-β-CD/EO₇₅PO₃₀EO₇₅ (5 mL), a manner in which the β-CD/EO₇₅PO₃₀EO₇₅ remained in the membrane without passing the pores was able to be recognized. In the pore-β-CD/EO₇₅PO₃₀EO₇₅ (20 mL), a manner in which the pores were completely covered with the β-CD/EO₇₅PO₃₀EO₇₅ was able to be recognized.

In the pore-β-CD/EO₇₅PO₃₀EO₇₅ (20 mL), the manner in which the pores were completely covered with the β-CD/EO₇₅PO₃₀EO₇₅ was able to be recognized. Accordingly, next, a sample for the evaluation of oxygen permeability was produced by using the pore-β-CD/EO₇₅PO₃₀EO₇₅ (20 mL). A production method is as described below. A nitrile rubber was dissolved in an organic solvent at a concentration of 2 wt %, and the pore-β-CD/EO₇₅PO₃₀EO₇₅ (20 mL) was immersed in the solution. After that, the sample was removed, and was naturally dried at room temperature to produce a nitrile rubber film carrying the pore-β-CD/EO₇₅PO₃₀EO₇₅ (20 mL).

The oxygen permeability of the nitrile rubber film carrying the pore-β-CD/EO₇₅PO₃₀EO₇₅ (20 mL) was measured. As a result, the oxygen permeability coefficient thereof was 18.6 cc·mm/(m²·day·atm). The oxygen permeability coefficient was suppressed to about 1/300 of the value of a typical nitrile rubber film (18.6 cc·mm/(m²·day·atm)), and hence it was found that the β-CD/EO₇₅PO₃₀EO₇₅ had an oxygen barrier property. An apparatus for measuring the oxygen permeability coefficient and conditions for the measurement are as described below.

Measuring apparatus: A MOCON (trademark) coulometric oxygen permeability-measuring apparatus (OX-TRAN (trademark) 2/22L)
Detector: A self-humidifying coulometric sensor
Corresponding standards: JIS K7126-2 (Plastic-Film and Sheet-Gas Permeability Test

### Method-Part II: Equal Pressure Method) (ISO 15105-2), and ASTM D3985, F1927, and F1307

Measurement temperature: 23° C.
Relative humidity: 0%
Effective membrane area: 1 cm²

### Example 18 Incorporation of Drug into Sheet-Shaped Nanostructure

### Production Example 1 Encapsulation 1 of Anti-Inflammatory Drug (Betamethasone) in Microstructure

The preparation of a betamethasone-encapsulated microstructure is as described below. 30 mg of PPO 4k (weight-average molecular weight: 4k: obtained from Sigma-Aldrich Co. LLC) and 10 mg of betamethasone (obtained from FUJIFILM Wako Pure Chemical Corporation) were dissolved in 1 mL of deionized water (having a pH of about 7) at 23° C.±1° C. Next, 120 mg of γ-CD (obtained from CycloChem Co., Ltd.) was added to 1 mL of the aqueous solution of the PPO 4k and betamethasone. The mixed solution was stirred with a vortex for 1 minute. Next, the solution was placed on a shaker at room temperature, and the solution was shaken to be aged. 30 Minutes after the shaking, the solution started to become lightly opaque, and 2 hours thereafter, the solution became more opaque. The solution was continuously shaken as it was at room temperature for 5 days.

Next, 800 μL of the resultant opaque liquid was loaded into a 1.5-milliliter centrifuge tube, and was centrifuged with a centrifugal separator (manufactured by Kenis Limited) at 12,000 rpm for 5 minutes. As a result, a white solid paste precipitated at the bottom of the centrifuge tube. 600 μL of the transparent supernatant was removed from the centrifuge tube, and 800 μL of a γ-CD aqueous solution (γ-CD concentration: 30 mg/mL) was newly added to the remaining white solid paste to suspend the white solid paste. The suspension was centrifuged again, and 800 μL of the supernatant was removed. The series of steps including the addition of the γ-CD aqueous solution, the centrifugation, and the removal of the supernatant was defined as a drug-washing step, and the drug-washing step was repeated five times.

The white solid paste after the drug washing was freeze-dried to provide 95 mg of a white solid.

The resultant white solid is referred to as “microstructure-1.”

The amount of betamethasone encapsulated in the microstructure-1 was calculated by absorbance measurement. 25 mg of the microstructure-1 was precisely weighed, and was dissolved in 1 mL of DMSO. The absorbance of the resultant DMSO solution was measured with a spectrophotometer (through use of a quartz cell having an optical path length of 1 mm), and the betamethasone concentration thereof was calculated. The calculation was performed as follows: the concentration-absorbance calibration curve of the solution of betamethasone in DMSO at a predetermined wavelength (λ=260 nm) was produced in advance; and the betamethasone concentration was calculated from the measured absorbance value and the calibration curve. The betamethasone concentration was calculated to be 1.0 mg/mL. 1.0 mg of betamethasone was encapsulated in 25 mg of the microstructure-1, and hence the betamethasone encapsulation ratio of the microstructure-1 was 4.0 mass %.

### Production Example 2 Encapsulation 2 of Betamethasone in Microstructure

Betamethasone was encapsulated in a microstructure by the same process as that of Production Example 1 except that PEO 4k (weight-average molecular weight: 4k: obtained from Sigma-Aldrich Co. LLC) was used instead of the PPO 4k as an axial molecule. The drug was washed five times, and the resultant white solid paste was freeze-dried to provide 88 mg of a white solid. The resultant white solid is referred to as “microstructure-2.” The betamethasone encapsulation ratio of the microstructure-2 was 8.8 mass %.

### Production Example 3 Encapsulation 1 of Diuretic (Spironolactone) in Microstructure

Spironolactone was encapsulated in a microstructure by the same process as that of Production Example 1 except that spironolactone (obtained from Tokyo Chemical Industry Co., Ltd.) was used instead of betamethasone as a drug. The drug was washed five times, and the resultant white solid paste was freeze-dried to provide 90 mg of a white solid. The resultant white solid is referred to as “microstructure-3.” The spironolactone encapsulation ratio of the microstructure-3 was 7.4 mass %.

### Production Example 4 Encapsulation 2 of Spironolactone in Microstructure

Spironolactone was encapsulated in a microstructure by the same process as that of Production Example 3 except that PEO 4k was used instead of the PPO 4k as an axial molecule. The drug was washed five times, and the resultant white solid paste was freeze-dried to provide 80 mg of a white solid. The resultant white solid is referred to as “microstructure-4.” The spironolactone encapsulation ratio of the microstructure-4 was 10.1 mass %.

### Production Example 5 Encapsulation 1 of Anti-Inflammatory Drug (Hydrocortisone) in Microstructure

Hydrocortisone was encapsulated in a microstructure by the same process as that of Production Example 1 except that hydrocortisone (obtained from Tokyo Chemical Industry Co., Ltd.) was used instead of betamethasone as a drug. The drug was washed five times, and the resultant white solid paste was freeze-dried to provide 78 mg of a white solid. The resultant white solid is referred to as “microstructure-5.” The hydrocortisone encapsulation ratio of the microstructure-5 was 3.6 mass %.

### Production Example 6 Encapsulation 2 of Hydrocortisone in Microstructure

Hydrocortisone was encapsulated in a microstructure by the same process as that of Production Example 5 except that PEO 4k was used instead of the PPO 4k as an axial molecule. The drug was washed five times, and the resultant white solid paste was freeze-dried to provide 83 mg of a white solid. The resultant white solid is referred to as “microstructure-6.” The hydrocortisone encapsulation ratio of the microstructure-6 was 5.8 mass %.

### Production Example 7 Encapsulation 1 of Anti-Inflammatory Drug (Dexamethasone) in Microstructure

Dexamethasone was encapsulated in a microstructure by the same process as that of Production Example 1 except that: dexamethasone (obtained from Tokyo Chemical Industry Co., Ltd.) was used instead of betamethasone as a drug; and the concentration-absorbance calibration curve of the drug was produced on the basis of the absorbance value thereof at a wavelength 2 of 270 nm. The drug was washed five times, and the resultant white solid paste was freeze-dried to provide a white solid. The resultant white solid is referred to as “microstructure-7.” The dexamethasone encapsulation ratio of the microstructure-7 was 2.3 mass %.

### Production Example 8 Encapsulation 2 of Dexamethasone in Microstructure

Dexamethasone was encapsulated in a microstructure by the same process as that of Production Example 7 except that PEO 4k was used instead of the PPO 4k as an axial molecule. The drug was washed five times, and the resultant white solid paste was freeze-dried to provide 68 mg of a white solid. The resultant white solid is referred to as “microstructure-8.” The dexamethasone encapsulation ratio of the microstructure-8 was 8.3 mass %.

### Production Example 9 Encapsulation 1 of Anticancer Drug (Doxorubicin Hydrochloride) in Microstructure

Doxorubicin hydrochloride was encapsulated in a microstructure by the same process as that of Production Example 1 except that: doxorubicin hydrochloride (obtained from Tokyo Chemical Industry Co., Ltd.) was used instead of betamethasone as a drug; and the concentration-absorbance calibration curve of the drug was produced on the basis of the absorbance value thereof at a wavelength 2 of 500 nm. The drug was washed four times, and the resultant white solid paste was freeze-dried to provide 80 mg of a white solid. The resultant white solid is referred to as “microstructure-9.” The doxorubicin hydrochloride encapsulation ratio of the microstructure-9 was 0.2 mass %.

### Production Example 10 Encapsulation 2 of Anticancer Drug (Doxorubicin Hydrochloride) in Microstructure

Doxorubicin hydrochloride was encapsulated in a microstructure by the same process as that of Production Example 9 except that PPO 2k (weight-average molecular weight: 2k: obtained from Sigma-Aldrich Co. LLC) was used instead of the PPO 4k as an axial molecule. The drug was washed four times, and the resultant white solid paste was freeze-dried to provide 75 mg of a white solid. The resultant white solid is referred to as “microstructure-10.” The doxorubicin hydrochloride encapsulation ratio of the microstructure-10 was 0.2 mass %.

### Production Example 11 Encapsulation 3 of Anticancer Drug (Doxorubicin Hydrochloride) in Microstructure

Doxorubicin hydrochloride was encapsulated in a microstructure by the same process as that of Production Example 9 except that PPO 0.7k (weight-average molecular weight: 0.7k: obtained from FUJIFILM Wako Pure Chemical Corporation) was used instead of the PPO 4k as an axial molecule. The drug was washed five times, and the resultant white solid paste was freeze-dried to provide 50 mg of a white solid. The resultant white solid is referred to as “microstructure-11.” The doxorubicin hydrochloride encapsulation ratio of the microstructure-11 was 0.2 mass %.

### Production Example 12 Encapsulation 4 of Anticancer Drug (Doxorubicin Hydrochloride) in Microstructure

Doxorubicin hydrochloride was encapsulated in a microstructure by the same process as that of Production Example 9 except that PEO 4k was used instead of the PPO 4k as an axial molecule. The drug was washed five times, and the resultant white solid paste was freeze-dried to provide 81 mg of a white solid. The resultant white solid is referred to as “microstructure-12.” The doxorubicin hydrochloride encapsulation ratio of the microstructure-12 was 0.3 mass %.

### Production Example 13 Encapsulation of Drug for Glaucoma Treatment (Dorzolamide Hydrochloride) in Microstructure

Dorzolamide hydrochloride was encapsulated in a microstructure by the same process as that of Production Example 12 except that: dorzolamide hydrochloride (obtained from Tokyo Chemical Industry Co., Ltd.) was used instead of doxorubicin hydrochloride as a drug; and the concentration-absorbance calibration curve of the drug was produced on the basis of the absorbance value thereof at a wavelength 2 of 260 nm. The drug was washed five times, and the resultant white solid paste was freeze-dried to provide 88 mg of a white solid. The resultant white solid is referred to as “microstructure-13.” The dorzolamide hydrochloride encapsulation ratio of the microstructure-13 was 1.3 mass %.

### Production Example 14 Encapsulation of Drug for Dementia Treatment (Donepezil Hydrochloride) in Microstructure

Donepezil hydrochloride was encapsulated in a microstructure by the same process as that of Production Example 12 except that: donepezil hydrochloride (obtained from Tokyo Chemical Industry Co., Ltd.) was used instead of doxorubicin hydrochloride as a drug; and the concentration-absorbance calibration curve of the drug was produced on the basis of the absorbance value thereof at a wavelength λ of 270 nm. The drug was washed four times, and the resultant white solid paste was freeze-dried to provide 70 mg of a white solid. The resultant white solid is referred to as “microstructure-14.” The donepezil hydrochloride encapsulation ratio of the microstructure-14 was 1.5 mass %.

### Production Example 15 Encapsulation 2 of Donepezil Hydrochloride in Microstructure

Donepezil hydrochloride was encapsulated in a microstructure by the same process as that of Production Example 14 except that PEO 4k was used instead of the PPO 4k as an axial molecule. The drug was washed five times, and the resultant white solid paste was freeze-dried to provide 91 mg of a white solid. The resultant white solid is referred to as “microstructure-15.” The donepezil hydrochloride encapsulation ratio of the microstructure-15 was 1.0 mass %.

### Production Example 16 Encapsulation of Anticancer Drug (Nimustine Hydrochloride) in Microstructure

Nimustine hydrochloride was encapsulated in a microstructure by the same process as that of Production Example 12 except that: nimustine hydrochloride (obtained from Tokyo Chemical Industry Co., Ltd.) was used instead of doxorubicin hydrochloride as a drug; and the concentration-absorbance calibration curve of the drug was produced on the basis of the absorbance value thereof at a wavelength λ of 270 nm. The drug was washed five times, and the resultant white solid paste was freeze-dried to provide 58 mg of a white solid. The resultant white solid is referred to as “microstructure-16.” The nimustine hydrochloride encapsulation ratio of the microstructure-16 was 0.2 mass %.

### Production Example 17 Encapsulation of Anticancer Drug (Dacarbazine) in Microstructure

Dacarbazine was encapsulated in a microstructure by the same process as that of Production Example 12 except that: dacarbazine (obtained from Tokyo Chemical Industry Co., Ltd.) was used instead of doxorubicin hydrochloride as a drug; and the concentration-absorbance calibration curve of the drug was produced on the basis of the absorbance value thereof at a wavelength λ of 350 nm. The drug was washed five times, and the resultant white solid paste was freeze-dried to provide 69 mg of a white solid. The resultant white solid is referred to as “microstructure-17.” The dacarbazine encapsulation ratio of the microstructure-17 was 0.5 mass %.

### Production Example 18 Encapsulation of Anticancer Drug (Cytarabine) in Microstructure

Cytarabine was encapsulated in a microstructure by the same process as that of Production Example 12 except that: cytarabine (obtained from Tokyo Chemical Industry Co., Ltd.) was used instead of doxorubicin hydrochloride as a drug; and the concentration-absorbance calibration curve of the drug was produced on the basis of the absorbance value thereof at a wavelength λ of 280 nm. The drug was washed five times, and the resultant white solid paste was freeze-dried to provide 93 mg of a white solid. The resultant white solid is referred to as “microstructure-18.” The cytarabine encapsulation ratio of the microstructure-18 was 0.1 mass %.

### Production Example 19 Encapsulation 1 of Clofibrate in Microstructure

Clofibrate was encapsulated in a microstructure by the same process as that of Production Example 1 except that: clofibrate (obtained from Tokyo Chemical Industry Co., Ltd.) was used instead of betamethasone as a drug; a solution (γ-CD concentration: 30 mg/mL) of γ-CD in a mixed solvent (water/ethanol=50 vol %/50 vol %) was used as a washing liquid in the drug-washing step; and the concentration-absorbance calibration curve of the drug was produced on the basis of the absorbance value thereof at a wavelength λ of 280 nm. The drug was washed five times, and the resultant white solid paste was freeze-dried to provide a white solid. The resultant white solid is referred to as “microstructure-19.” The clofibrate encapsulation ratio of the microstructure-19 was 6.7 mass %.

### Production Example 20 Encapsulation 2 of Clofibrate in Microstructure

Clofibrate was encapsulated in a microstructure by the same process as that of Production Example 15 except that PEO 4k was used instead of the PPO 4k as an axial molecule. The drug was washed five times, and the resultant white solid paste was freeze-dried to provide a white solid. The resultant white solid is referred to as “microstructure-20.” The clofibrate encapsulation ratio of the microstructure-20 was 2.9 mass %.

### Production Example 21 Encapsulation 1 of Anticancer Drug (5-Fluorouracil) in Microstructure

5-Fluorouracil was encapsulated in a microstructure by the same process as that of Production Example 15 except that: 5-fluorouracil (obtained from Tokyo Chemical Industry Co., Ltd.) was used instead of betamethasone as a drug; and the concentration-absorbance calibration curve of the drug was produced on the basis of the absorbance value thereof at a wavelength λ of 270 nm. The drug was washed five times, and the resultant white solid paste was freeze-dried to provide a white solid. The resultant white solid is referred to as “microstructure-21.” The 5-fluorouracil encapsulation ratio of the microstructure-21 was 0.4 mass %.

### Production Example 22 Encapsulation 2 of 5-Fluorouracil in Microstructure

5-Fluorouracil was encapsulated in a microstructure by the same process as that of Production Example 17 except that PEO 4k was used instead of the PPO 4k as an axial molecule. The drug was washed four times, and the resultant white solid paste was freeze-dried to provide a white solid. The resultant white solid is referred to as “microstructure-22.” The 5-fluorouracil encapsulation ratio of the microstructure-22 was 0.1 mass %.

### Production Example 23 Encapsulation 1 of Anticancer Drug (Docetaxel) in Microstructure

Docetaxel was encapsulated in a microstructure by the same process as that of Production Example 15 except that: docetaxel (obtained from Tokyo Chemical Industry Co., Ltd.) was used instead of betamethasone as a drug; and the concentration-absorbance calibration curve of the drug was produced on the basis of the absorbance value thereof at a wavelength λ of 285 nm. The drug was washed five times, and the resultant white solid paste was freeze-dried to provide a white solid. The resultant white solid is referred to as “microstructure-23.” The docetaxel encapsulation ratio of the microstructure-23 was 0.5 mass %.

### Production Example 24 Encapsulation 2 of Docetaxel in Microstructure

Docetaxel was encapsulated in a microstructure by the same process as that of Production Example 19 except that PEO 4k was used instead of the PPO 4k as an axial molecule. The drug was washed five times, and the resultant white solid paste was freeze-dried to provide a white solid. The resultant white solid is referred to as “microstructure-24.” The docetaxel encapsulation ratio of the microstructure-24 was 2.9 mass %.

TABLE 1

| | | | | Weight of |
|---|---|---|---|---|
| | | | | encap- |
| | | Microstructure | | sulated |
| | | composition | | drug/ |
| | | Cyclic | | weight of |
| | Encapsulated | mole- | Axial | micro- |
| Production Example | drug | cule | polymer | structure |
| Production Example 1 | Betamethasone | γ-CD | PPO 4k | 4.0% |
| Production Example 2 | Betamethasone | γ-CD | PEO 4k | 8.8% |
| Production Example 3 | Spironolactone | γ-CD | PPO 4k | 7.4% |
| Production Example 4 | Spironolactone | γ-CD | PEO 4k | 10.1% |
| Production Example 5 | Hydrocortisone | γ-CD | PPO 4k | 3.6% |
| Production Example 6 | Hydrocortisone | γ-CD | PEO 4k | 5.8% |
| Production Example 7 | Dexamethasone | γ-CD | PPO 4k | 2.3% |
| Production Example 8 | Dexamethasone | γ-CD | PEO 4k | 8.3% |
| Production Example 9 | Doxorubicin | γ-CD | PPO 4k | 0.2% |
| | hydrochloride | | | |
| Production Example 10 | Doxorubicin | γ-CD | PPO 2k | 0.2% |
| | hydrochloride | | | |
| Production Example 11 | Doxorubicin | γ-CD | PPO 0.7k | 0.2% |
| | hydrochloride | | | |
| Production Example 12 | Doxorubicin | γ-CD | PEO 4k | 0.3% |
| | hydrochloride | | | |
| Production Example 13 | Dorzolamide | γ-CD | PEO 4k | 1.3% |
| | hydrochloride | | | |
| Production Example 14 | Donepezil | γ-CD | PPO 4k | 1.5% |
| | hydrochloride | | | |
| Production Example 15 | Donepezil | γ-CD | PEO 4k | 1.0% |
| | hydrochloride | | | |
| Production Example 16 | Nimustine | γ-CD | PPO 4k | 0.2% |
| | hydrochloride | | | |
| Production Example 17 | Dacarbazine | γ-CD | PPO 4k | 0.5% |
| Production Example 18 | Cytarabine | γ-CD | PPO 4k | 0.1% |
| Production Example 19 | Clofibrate | γ-CD | PPO 4k | 6.7% |
| Production Example 20 | Clofibrate | γ-CD | PEO 4k | 2.9% |
| Production Example 21 | 5-Fluorouracil | γ-CD | PPO 4k | 0.4% |
| Production Example 22 | 5-Fluorouracil | γ-CD | PEO 4k | 0.1% |
| Production Example 23 | Docetaxel | γ-CD | PPO 4k | 0.5% |
| Production Example 24 | Docetaxel | γ-CD | PEO 4k | 2.9% |

### Example 19 Incorporation of Fluorescent Molecule into Sheet-Shaped Nanostructure

### Production Example 25 Encapsulation of Fluorescent Molecule (Fluorescein Isothiocyanate (FITC)) in Microstructure

FITC was encapsulated in a microstructure by the same process as that of Production Example 1 except that: FITC that was a fluorescent molecule was used instead of betamethasone serving as a drug; and the concentration-absorbance calibration curve of the fluorescent molecule was produced on the basis of the absorbance value thereof at a wavelength λ of 280 nm. The fluorescent molecule was washed six times, and the resultant pale yellow solid paste was freeze-dried to provide 70 mg of a pale yellow solid. The resultant pale yellow solid is referred to as “microstructure-25.” The FITC encapsulation ratio of the microstructure-25 was 0.2 mass %.

In addition, 10 mg of the microstructure-25 was added to 200 μL of a γ-CD aqueous solution (γ-CD concentration: 30 mg/mL), and an ultrasonic wave was applied from an ultrasonic cleaner for 1 minute to disperse the microstructure in the solution. The resultant microstructure-dispersed liquid was observed with a fluorescence microscope (manufactured by Nikon Corporation: using Nikon DS-Fi3). Light having a wavelength of 470 nm was used as an excitation light in the fluorescence observation. Fluorescence emission was observed in the microstructure, and hence the encapsulation of FITC in the microstructure was recognized.

### Production Example 26 Encapsulation of Rhodamine B in Microstructure

Rhodamine B was encapsulated in a microstructure by the same process as that of Production Example 25 except that: rhodamine B was used instead of FITC as a fluorescent molecule; and PEO 20k was used instead of the PPO 4k as an axial molecule. The fluorescent molecule was washed six times, and the resultant reddish purple solid paste was freeze-dried to provide 66 mg of a pink solid. The resultant reddish purple solid is referred to as “microstructure-26.” The rhodamine B encapsulation ratio of the microstructure-26 was 0.1 mass %.

In addition, fluorescence microscope observation was performed by the same process as that of Example 25. Fluorescence emission was observed in the microstructure, and hence the encapsulation of rhodamine B in the microstructure was recognized.

### Production Example 27 Encapsulation of Uranine in Microstructure

Uranine was encapsulated in a microstructure by the same process as that of Production Example 25 except that: uranine was used instead of FITC as a fluorescent molecule; and PPO 2k was used instead of the PPO 4k as an axial molecule. The fluorescent molecule was washed six times, and the resultant reddish purple solid paste was freeze-dried to provide 75 mg of a pink solid. The resultant pink solid is referred to as “microstructure-27.” The uranine encapsulation ratio of the microstructure-27 was 0.1 mass %.

In addition, fluorescence microscope observation was performed by the same process as that of Example 25. Fluorescence emission was observed in the microstructure, and hence the encapsulation of uranine in the microstructure was recognized.

TABLE 2

| | | Microstructure | | Weight of |
|---|---|---|---|---|
| | Encapsulated | composition | | encapsulated |
| Production | fluorescent | Cyclic | Axial | drug/weight of |
| Example | molecule | molecule | polymer | microstructure |
| Production | FITC | γ-CD | PPO 4k | 0.2% |
| Example 25 | | | | |
| Production | Rhodamine B | γ-CD | PEO 20k | 0.1% |
| Example 26 | | | | |
| Production | Uranine | γ-CD | PPO 2k | 0.1% |
| Example 27 | | | | |

1. A structure comprising a plurality of pseudo-polyrotaxanes and/or polyrotaxanes each including a chain polymer included in a cavity or cavities of one or more cyclic molecules in a skewered manner, at least part of the plurality of pseudo-polyrotaxanes and/or polyrotaxanes being arranged in series with each other.

2. The structure according to claim 1, wherein the chain polymer is a single chain.

3. The structure according to claim 1, wherein the chain polymer is included in the cavities of the three or more cyclic molecules per one chain polymer in a skewered manner.

4. The structure according to claim 1, wherein at least part of the plurality of pseudo-polyrotaxanes and/or polyrotaxanes are arranged in parallel with each other.

5. The structure according to claim 1, further comprising a substance that is bonded to the chain polymer, is bonded to the cyclic molecule, or is held in one of a space between the plurality of pseudo-polyrotaxanes and/or polyrotaxanes, the cavity defined and formed by the one cyclic molecule, and a space defined and formed by the plurality of cyclic molecules.

6. The structure according to claim 5, wherein the substance is at least one selected from the group consisting of a drug, a fluorescent substance, and a chromogenic enzyme.

7. The structure according to claim 1, wherein the chain polymer has a length of from 1 nm to 2,000 nm.

8. The structure according to claim 1, wherein the chain polymer includes a moiety formed of one of polyethylene glycol (PEG) or polypropylene glycol (PPG).

9. The structure according to claim 1, wherein the cyclic molecule is at least one selected from the group consisting of a cyclodextrin, a crown ether, a pillararene, a calixarene, a cyclophane, and derivatives thereof.

10. The structure according to claim 1, wherein the structure has arranged therein at least one through-hole penetrating the structure and occupying a space larger than a volume occupied by the one pseudo-polyrotaxane and/or polyrotaxane.

11. The structure according to claim 1, wherein the structure is any one of the following (a) to (c):
(a) a rod-shaped structure in which the chain polymer has a weight-average molecular weight of from 200 to 10,000;
(b) a cube-shaped structure in which the chain polymer has a weight-average molecular weight of from 1,000 to 20,000; and
(c) a sheet-shaped structure in which the chain polymer has a weight-average molecular weight of from 2,000 to 200,000.

12. A method of producing a structure including a plurality of pseudo-polyrotaxanes and/or polyrotaxanes each including a chain polymer included in a cavity or cavities of one or more cyclic molecules in a skewered manner, the method comprising causing the plurality of pseudo-polyrotaxanes and/or polyrotaxanes each including the chain polymer having both ends housed in a column formed of the plurality of cyclic molecules to interact with each other to arrange at least part of the plurality of pseudo-polyrotaxanes and/or polyrotaxanes in series with each other.
